(19) United States
(12) Patent Application Publication
(10) Pub. No.: US 2013/0217017 A1
(43) Pub. Date: Aug. 22, 2013

(54) Detection and Assessment of Cancer Risk Using Telomere Health

(71) Applicant: Georgetown University, (US)

(72) Inventor: Yun-Ling Zheng, North Potomac, MD (US)

(21) Appl. No.: 13/843,936

(22) Filed: Mar. 15, 2013

Related U.S. Application Data
(63) Continuation-in-part of application No. PCT/US2011/056545, filed on Oct. 17, 2011.

(51) Int. Cl. C12Q 1/68

(52) U.S. Cl. CPC C12Q 1/6886
USPC 435/6.11

(57) ABSTRACT

Disclosed are compositions and methods related to assessing the risk of cancer, such as breast cancer and lung cancer, through analyzing the length of telomeres, such as chromosome 9p, 15p, and/or Xp telomere, such as the short arm of the 9p, 15p, and/or Xp telomere. For example, if the 9p, 15p, and/or Xp arm is shorter than normal, the risk of cancer is increased.

### CROSS-REFERENCE TO RELATED APPLICATIONS

This application is a continuation-in-part of International Application No. PCT/US2011/056545, filed Oct. 17, 2011. Application No. PCT/US2011/056545, filed Oct. 17, 2011, is hereby incorporated herein by reference in its entirety.

### STATEMENT REGARDING FEDERALLY SPONSORED RESEARCH OR DEVELOPMENT

This invention was made with government support under Grants P30 CA51008 and DAMD17-03-1-0446 awarded by the National Institutes of Health and Department of Defense, respectively. The government has certain rights in the invention.

### FIELD OF THE INVENTION

The present disclosure is directed generally to compositions and methods for detecting and assessing cancer risk using telomere health.

### BACKGROUND OF THE INVENTION

Breast cancer is the most common malignancy in women (Parkin et al. Breast J 12 Suppl 1:S70-S80, 2006). In the United States, breast cancer incidence rates have been rising slowly for the past two decades, and breast cancer is the second leading cause of cancer-related death in women (Stewart et al. MMWR 53:1-108, 2004; Smigal et al. CA Cancer J Clin 56:168-183, 2006). However, there is currently no accurate method to predict who is most likely to develop the disease for individuals in general population. Of the nearly 241,000 women diagnosed each year, about 80%-90% are sporadic cases who had no family history of breast cancer and no other identifiable strong risk factors other than age and reproductive or hormonal risk factors (Lancet 358:1389-1399, 2001). In order to prevent breast cancer, there is a need to develop tools to identify women at an elevated risk, allowing women and their physicians to take a more proactive approach to reduce breast cancer burden.

Cancer genomes are highly rearranged and are characterized by complex translocations and regional copy number alterations (Pinkel D et al, 2005, Nat Genet 37:S11-17). Efforts to uncover the underlying mechanisms driving chromosome instability in cancer have revealed a prominent role for telomeres (reviewed by Artandi et al 2010, Carcinogenesis, 31:9). Telomeres, the nucleoprotein complexes at the end of eukaryotic chromosomes, are specialized structures that protect chromosome ends and prevent them from being recognized by the cell as DNA double-strand breaks (Blackburn Cell 106(6):661-73, 2001). Telomeres are vulnerable due to progressive shortening during each round of DNA replication (Harley et al. Nature 345(6274):458-60, 1990; Olovnikov, J Theor Biol. 41(1):181-90, 1973; Watson, 1972) and telomere length is directly related to the proliferative history of the cell. Thus a lifetime of tissue renewal places the organism at risk for telomere dysfunction and increasing chromosomal instability, particularly in aged populations. Dysfunctional telomeres result in inappropriate chromosomal end-to-end fusions through the non-homologous end joining or homologous recombination DNA repair pathways (Verdun, Nature. 447(7147):924-31, 2007). These fusions are the basis of chromosome instability through repetition of breakage-fusion-bridge cycle, causing chromosome abnormalities that were typically seen in most human cancers (Gisselsson, PNAS. 98(22):12683-8, 2001; Murnane DNA Repair (Amst). 5(9-10):1082-92, 2006).

Although there is compelling evidence that telomere dysfunction (very short or extremely long telomeres) and chromosome instability are characteristics of breast tumors (Meeker, J Mammary Gland Biol Neoplasia. 9(3):285-96, 2004; Meeker, Am J. Pathol. 160(4):1259-68, 2002; Fridlyand, BMC Cancer. 6:96.2006), connections between telomeres and breast cancer risk has not been established. Several case-control studies have examined overall telomere length in blood leucocytes and risk of breast cancer (Zheng, 2009; Shen, 2007; Svenson, 2008; Shen, 2009; Barwell, 2007) and reported controversial results, with the majority of the studies (Zheng, 2009; Shen, 2007; Shen, 2009; Barwell, 2007) reporting no significant association. One of the major limitations of these previous studies is that only the overall telomere length (average telomere length of 92 telomeres in the human genome) was measured. Telomere lengths on each chromosome end were not assessed.

A key feature necessary for telomere integrity is the maintenance of telomeric DNA at a critical length that allows assembly of the protective end structures. Current knowledge indicates that telomere length are maintained by three distinct mechanisms: (1) replenishment of telomere DNA by telomerase (a ribonucleoprotein reverse transcriptase) (Greider, 1996); (2) alternative lengthening of telomeres (ALT) involving homologous recombination (Cesare aj, 2008, Mechanisms of Ageing, 129:99); (3) epigenetic modification of telomeric and subtelomeric DNA (Blasco, 2007). Telomeres are thought to play a key role in tumor suppression by limiting the number of times a cell can divide, even in the presence of oncogenic mutations. Because human somatic cells lack an active telomere length maintenance mechanism, telomeres shorten with each cell division due to the end replication problem (Harley, 1990; Olovnikov, 1973; Watson, 1972). In most normal somatic cells, the level of telomerase activity is either undetectable or detectable but insufficient to completely prevent telomere shortening. In addition to the predicted end-replication losses, telomeres can be subject to large-scale stochastic deletion events, rapidly creating telomeres that are very short and dysfunctional (Baird et al 2003, 33:203). Deficiencies in telomere length maintenance is particularly relevant to carcinogenesis because hyper-proliferative cancerous cells could lead to progressive telomere shortening, ultimately generating uncapped telomeres that fuse with each other leading to genomic instability that promotes malignant transformation. Only two small studies examined telomere length on a few chromosome arms and the risk of breast (Zheng, 2009) and esophageal (Xing, 2009) cancers. Short telomere length on chromosome 9p was found to be significantly associated with breast cancer risk (Zheng, 2009) and short telomeres on chromosome 17p and 12q were found to be significantly associated with an increased risk of esophageal cancer (Xing, 2009).

It is therefore an object of the present invention to provide a method for detecting and assessing risk of cancer.

It is also an object of the present invention to provide a method that uses telomere health to detect and assess risk of cancer.

### BRIEF SUMMARY OF THE INVENTION

Methods for detecting and assessing cancer risk are provided. It was discovered that telomere health is strongly associated with breast cancer risk and lung cancer risk. For example, individual telomere length and variation in telomere length, which indicate telomere health, are strongly associated with breast cancer risk. In some forms, measures of telomere health such as shorter telomeres on certain chromosomes, certain chromosome arms, one homolog of certain homologous chromosomes pairs, and one homolog of certain homologous chromosome arm pairs are associated with breast cancer risk. As another example, other measures of telomere health such as greater variation in telomere length among all, some, or certain chromosomes, chromosome arms, homologous chromosomes, and homologous chromosome arms are associated with breast cancer risk. Specific examples include significant association of breast cancer risk in premenopausal women with measures of telomere health such as short telomere length on chromosomes Xp and 15p, greater length differences between homologous telomeres on chromosomes 9p, 15p and 15q, greater telomere length variation in lymphocytes on chromosome 18p, and greater variation in telomere length among the chromosomes of a cell are.

For example, variation in telomere length and the frequency of extremely short telomeres, which indicate telomere health, are strongly associated with lung cancer risk. In some forms, greater telomere length variation, greater frequency of extremely short telomeres, or both, indicate a risk of lung cancer. In some forms, variation in telomere length and the frequency of extremely short telomeres, or both, in subjects 60 years of age or younger indicate a risk of lung cancer risk.

The data herein provide the first evidence that telomere health or deficiency on certain chromosome arms are linked to breast cancer and lung cancer susceptibility and risk. These new discoveries have clinical application in detecting and assessing cancer risk, the application of which is the subject of the disclosed methods. The disclosed telomere-related parameters can be used, for example, as a panel of blood-based biomarkers for cancer risk detection and assessment. The disclosed telomere health measures such as chromosome telomere length measurements and assessments can be incorporated into the current and future prediction and prognosis models to enhance breast cancer and lung cancer risk prediction and prognosis. The disclosed methods can be used to improve the efficiency of, for example, both population-based preventive programs, such as screening mammography or chest x-rays, and individual-based preventive strategies such as chemoprevention by targeting women who are at the greatest risk for breast cancer.

Disclosed are methods of assaying a subject comprising measuring parameters of the health of at least one chromosome telomere of a chromosome in at least one cell of a sample from a subject, thereby producing a chromosome telomere health for at least one of the chromosome telomeres, and comparing the chromosome telomere health with a reference chromosome telomere health. By measuring parameters of the health of individual chromosome telomeres and comparing to parameters of reference chromosome telomere healths, cancer risk in subjects can be assessed. Any or a combination of parameters of chromosome telomere health can be used for such measurements. In some forms, the parameter of the chromosome telomere health can be the length of the chromosome telomere. In some forms, the parameter of the reference chromosome telomere health can be a reference chromosome telomere length.

For example, pre-menopausal women have an increased risk of breast cancer if the chromosome telomere health of chromosome telomere 1p-S, Xp-S, 9p-S, or 15p-S is less than the reference chromosome telomere health. As another example, post-menopausal women have an increased risk of breast cancer if the chromosome telomere health of chromosome telomere 15p-S is less than the reference chromosome telomere health. As another example, pre-menopausal women have greater risk of breast cancer the shorter the chromosome telomere health of chromosome telomere Xp-S or 15p-S is less than the reference chromosome telomere health.

Reduced chromosome telomere health can be established using any parameter or degree of chromosome telomere health less than the reference chromosome telomere health. For example, the chromosome telomere health can be a fraction of the reference chromosome telomere health. For example, in the case where the parameter of chromosome telomere health is chromosome telomere length, the chromosome telomere length can be less than or equal to 0.5 of the reference chromosome telomere length (as the appropriate reference chromosome telomere health).

Differences in the parameters of chromosome telomere health of homologous telomeres (telomeres in homologous chromosome arms) can also be used to assess a subject's risk of cancer. For example, pre-menopausal women have an increased risk of breast cancer if the homologous telomere length difference (HTLD) is less in chromosome arm 5q, Xp, 8q, 9p, 12p, 15p, or 15q than a reference HTLD, such as the HTLD for the chromosome arm in normal subjects. As another example, pre-menopausal women have a greater risk of breast cancer the lower the HTLD in chromosome arm Xp, 9p, 15p, or 15q. Any or a combination of parameters of chromosome telomere health can be used for such measurements. As another example, the subject has an increased risk of breast cancer if the homologous telomere length difference (HTLD) is greater in chromosome arm 5q, Xp, 8q, 9p, 12p, 15p, or 15q than a reference homologous telomere length difference (HTLD). The reference HTLD can be, for example, the average, median, or quartile value of the HTLDs in cells from normal subjects of similar type to the cell.

The level of variability in telomere health within a cell (such as between the telomeres of a cell) can also be used to assess cancer risk. For example, pre-menopausal women have an increased risk of breast cancer if the within-cell telomere length variation (WCTLV) is less than a reference WCTLV, such as the WCTLV in normal subjects. As another example, pre-menopausal female have greater risk of breast cancer the lower the WCTLV. Any or a combination of parameters of chromosome telomere health can be used for such assessments. As another example, the subject has an increased risk of breast cancer if the within-cell telomere length variation (WCTLV) is greater than a reference within-cell telomere length variation (WCTLV). The reference WCTLV can be, for example, the average, median, or quartile value of the WCTLV in cells from normal subjects of similar type to the cell.

For these methods, it is useful to use the relative telomere health as the chromosome telomere health. Reference chromosome telomere healths preferably can be those of normal controls. In general, such normal controls can be similar chromosome telomere health parameter measurements made in unaffected subjects and cells. For example, the reference chromosome telomere health can be the average, median, or quartile value of the chromosome telomere health in cells from normal subjects of similar type to the cell, the average, median, or quartile value of the chromosome telomere health of the chromosome in cells from normal subjects of similar type to the cell, or the average, median, or quartile value of the arm-specific telomere health in cells from normal subjects of similar type to the cell. Any or a combination of parameters of chromosome telomere health can be used for such measurements. In some forms, the parameter of the chromosome telomere health can be the relative telomere length.

Also disclosed are methods of assaying a subject comprising measuring the length of at least one chromosome telomere of a chromosome in at least one cell of a sample from a subject, thereby producing a chromosome telomere length for at least one of the chromosome telomeres, and comparing the chromosome telomere length with a reference chromosome telomere length. By measuring individual chromosome telomeres and comparing to reference chromosome telomere lengths, cancer risk in subjects can be assessed.

For example, pre-menopausal women have an increased risk of breast cancer if the chromosome telomere length of chromosome telomere 1p-S, Xp-S, 9p-S, or 15p-S is shorter than the reference chromosome telomere length. As another example, post-menopausal women have an increased risk of breast cancer if the chromosome telomere length of chromosome telomere 15p-S is shorter than the reference chromosome telomere length. As another example, pre-menopausal women have greater risk of breast cancer the shorter the chromosome telomere length of chromosome telomere Xp-S or 15p-S is shorter than the reference chromosome telomere length.

Shorter chromosome telomere lengths can be any length or degree shorter than the reference chromosome telomere length. For example, the chromosome telomere length can be a fraction of the reference chromosome telomere length. For example, the chromosome telomere length can be less than or equal to 0.5 of the reference chromosome telomere length.

Differences in the length of homologous telomeres (telomeres in homologous chromosome arms) can also be used to assess a subject's risk of cancer. For example, pre-menopausal women have an increased risk of breast cancer if the homologous telomere length difference (HTLD) is greater in chromosome arm 5q, Xp, 8q, 9p, 12p, 15p, or 15q than a reference HTLD, such as the HTLD for the chromosome arm in normal subjects. As another example, pre-menopausal women have a greater risk of breast cancer the greater the HTLD in chromosome arm Xp, 9p, 15p, or 15q. As another example, the subject has an increased risk of breast cancer if the homologous telomere length difference (HTLD) is greater in chromosome arm 5q, Xp, 8q, 9p, 12p, 15p, or 15q than a reference homologous telomere length difference (HTLD). The reference HTLD can be, for example, the average, median, or quartile value of the HTLDs in cells from normal subjects of similar type to the cell.

The level of variability in telomere length within a cell (such as between the telomeres of a cell) can also be used to assess cancer risk. For example, pre-menopausal women have an increased risk of breast cancer if the within-cell telomere length variation (WCTLV) is greater than a reference WCTLV, such as the WCTLV in normal subjects. As another example, pre-menopausal female have greater risk of breast cancer the greater the WCTLV. As another example, the subject has an increased risk of breast cancer if the within-cell telomere length variation (WCTLV) is greater than a reference within-cell telomere length variation (WCTLV). The reference WCTLV can be, for example, the average, median, or quartile value of the WCTLV in cells from normal subjects of similar type to the cell.

Variation in telomere length and the frequency of extremely short telomeres, which indicate telomere health, are strongly associated with lung cancer risk. In some forms, greater telomere length variation, greater frequency of extremely short telomeres, or both, indicate a risk of lung cancer. In some forms, variation in telomere length and the frequency of extremely short telomeres, or both, in subjects 60 years of age or younger indicate a risk of lung cancer risk.

In some forms, telomere length variation over median telomere length variation, frequency of extremely short telomeres over median frequency of extremely short telomeres, or both, indicate a risk of lung cancer. In some forms, telomere length variation over median telomere length variation, frequency of extremely short telomeres over median frequency of extremely short telomeres, or both, in subjects 60 years of age or younger indicate a risk of lung cancer.

In some forms, telomere length variation in the highest quartile of telomere length variation, frequency of extremely short telomeres in the highest quartile of frequency of extremely short telomeres, or both, indicate a risk of lung cancer. In some forms, telomere length variation in the highest quartile of telomere length variation, frequency of extremely short telomeres in the highest quartile of frequency of extremely short telomeres, or both, in subjects 60 years of age or younger indicate a risk of lung cancer.

For these methods, it is useful to use the relative telomere length as the chromosome telomere length. Reference chromosome telomere lengths preferably can be normal controls. In general, such normal controls can be similar chromosome telomere length measurements made in unaffected subjects and cells. For example, the reference chromosome telomere length can be the average, median, or quartile value of the chromosome telomere lengths in cells from normal subjects of similar type to the cell, the average, median, or quartile value of the chromosome telomere lengths of the chromosome in cells from normal subjects of similar type to the cell, or the average, median, or quartile value of the arm-specific telomere lengths in cells from normal subjects of similar type to the cell.

Any suitable techniques can be used to measure parameters of chromosome telomere health. Any suitable techniques can be used to measure the length of chromosome telomeres. For example, chromosome telomere length can be measured by obtaining the sample from the subject, where the sample is a blood sample, for example; harvesting the chromosome from at least one cell in the blood sample; performing telomere analysis; and quantitating telomere length. Telomere analysis and quantitating telomere length can also be accomplished using any suitable techniques. For example, telomere analysis and quantitating telomere length can be accomplished by telomere quantitative fluorescent in situ hybridization (QT-FISH). Useful for quantitating telomere length are techniques that total the fluorescent signal from telomere probes from the chromosome telomere of interest.

In accordance with the purpose of this invention, as embodied and broadly described herein, this invention relates to methods for assessing cancer risk based on parameters related to chromosomal telomere health.

Additional advantages of the disclosed methods will be set forth in part in the description which follows, and in part will be understood from the description, or may be learned by practice of the disclosed methods. The advantages of the disclosed methods will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION OF THE INVENTION

### I. Definitions

“Telomere health” or “chromosome telomere health” or like terms refers to one or more of a group of parameters that measure telomere health, including, for example, telomere length, telomere length variation among two or more telomeres, and frequency of extremely short or long telomeres.

“Overall telomere health” or like terms refers to a group of parameters that measure overall telomere health, including total telomere length, telomere length variation among the total telomeres in a typical cell of the type being assessed (there is a total of 92 chromosomal arms in a typical human cell, for example), and frequency of extremely short or long telomeres in a typical cell.

“Chromosome arm-specific telomere health” or like terms refers to one or more of a group of parameters that measure telomere health at a specific chromosome arm (there is a total of 92 chromosomal arms in a typical human cell), including, for example, telomere length at a specific chromosome arm, homologous telomere length difference, telomere length variation at a specific chromosome arm, and frequency of extremely short or long telomeres at a specific chromosome arm among a group of cells.

“Reference chromosome telomere health” or “reference telomere health” or like terms refers to a reference health of the chromosome telomere.

“Telomere length” (TL) or “chromosome telomere length” or “absolute telomere length (ATL) or like terms refers to the direct or indirect length of a telomere of a chromosome arm. “Total telomere length” or like terms refers to the length, direct or indirect, or all of the telomeres in a cell. In the case of human cells, there are a total of 92 chromosomal arms in a typical human cell. As used herein, length can be, for example, absolute length or an indirect measurement of length as discussed herein.

“Relative telomere health” (RTH) or “telomere health ratio” or like terms refers to a ratio between the health of at least one telomere of one arm of a chromosome in a cell and the health of the reference nucleic acid sequences, such as the health of all the telomeres of a complete set of chromosome arms (N=92) in a typical human cell. Thus, for example, a telomere health ratio could be parameter of chromosome telomere health from the short arm of chromosome X (that is Xp) to that parameter of chromosome telomere health of a reference nucleic acid sequence, that is, for example, the parameter of chromosome telomere health from the telomeres of the 92 arms of the chromosomes from a typical human cell. This would be a chromosome Xp relative telomere health or chromosome Xp telomere health ratio.

“Relative telomere length” (RTL) or “telomere ratio” or like terms refers to a ratio between the length of at least one telomere of one arm of a chromosome in a cell and the length of the reference nucleic acid sequences, such as the length of all the telomeres of a complete set of chromosome arms (N=92) in a typical human cell or the length of centromeric sequences of chromosome 2, etc. Thus, for example, a telomere ratio could be the signal from the short arm of chromosome X (that is Xp) to the signal of a reference nucleic acid sequence, that is, for example, signals from the telomeres of the 92 arms of the chromosomes from a typical human cell. This would be a chromosome Xp relative telomere length or chromosome Xp telomere ratio.

“Telomere length at a specific chromosome arm” or “arm-specific telomere length” or “Chromosome arm-specific telomere length” or like terms refers to the telomere length of either the p or q arm of a chromosome. In some instances it can be the telomere length of both homologous telomeres of the chromosome. In some instances it can be the telomere length of both the p and q arms of a chromosome. “Chromosome-specific telomere length” or like terms refers to the telomere length of both arms of a chromosome.

“Reference chromosome telomere length” or “reference telomere length” or like terms refers to a reference length of the chromosome telomere.

“Homologous telomere length difference” (HTLD) refers to (a) the telomere length of the longer telomere of a homologous pair of telomeres (TLhL) minus (b) the telomere length of the shorter telomere of the homologous pair of telomeres (TLhS), the result divided by the sum of (a) the telomere length of the longer telomere of the homologous pair of telomeres (TLhL) and (b) the telomere length of the shorter telomere of the homologous pair of telomeres (TLhS). This formula can be written using abbreviations as follows: HTLD=(TLhL−TLhS)/(TLhL+TLhS). Thus, for example, an HTLD for chromosome arm 15p would be the telomere length of the longer telomere of the homologous pair of 15p telomeres (15p TLhL) minus the telomere length of the shorter telomere of the homologous pair of 15p telomeres (15p TLhS), the result divided by the sum of the telomere length of the longer telomere of the homologous pair of 15p telomeres (15p TLhL) and the telomere length of the shorter telomere of the homologous pair of 15p telomeres (15p TLhS). The resulting HTLD can be referred to as a chromosome 15p HTLD. HTLD can be expressed as, for example, a fraction or percentage. “Reference HTLD” or like terms refers to a HTLD established from a sample(s) from a subject(s) that is considered a control. A reference HTLD could be, for example, from healthy individuals or from non-cancerous patients. It is understood that the reference HTLD can be produced de novo or can be a number previously determined as a reference length.

Homologous telomere length difference (HTLD) can use absolute telomere length or relative telomere length. For example, HTLD using absolute telomere length can be (a) the absolute telomere length of the longer telomere of a homologous pair of telomeres (ATLhL) minus (b) the absolute telomere length of the shorter telomere of the homologous pair of telomeres (ATLhS), the result divided by the sum of (a) the absolute telomere length of the longer telomere of the homologous pair of telomeres (ATLhL) and (b) the absolute telomere length of the shorter telomere of the homologous pair of telomeres (ATLhS). This formula can be written using abbreviations as follows: HTLD=(ATLhL−ATLhS)/(ATLhL+ATLhS). HTLD using absolute telomere length can be referred to as AHTLD. HTLD using relative telomere length can be (a) the relative telomere length of the longer telomere of a homologous pair of telomeres (RTLhL) minus (b) the relative telomere length of the shorter telomere of the homologous pair of telomeres (RTLhS), the result divided by the sum of (a) the relative telomere length of the longer telomere of the homologous pair of telomeres (RTLhL) and (b) the relative telomere length of the shorter telomere of the homologous pair of telomeres (RTLhS). This formula can be written using abbreviations as follows: HTLD=(RTLhL−RTLhS)/(RTLhL+RTLhS). HTLD using absolute telomere length can be referred to as RHTLD.

“Overall telomere length variation” or like terms refers to the coefficient of variation (CV) of the telomere lengths among all of the chromosome arms in a cell. Overall telomere length variation is thus a measure of the variability of telomere lengths in a cell. “Reference overall telomere length variation” or like terms refers to an overall telomere length variation established from a sample(s) from a subject(s) that is considered a control. A reference overall telomere length variation could be, for example, from healthy individuals or from non-cancerous patients. It is understood that the reference overall telomere length variation can be produced de novo or can be a number previously determined as a reference length.

“Telomere length variation at a specific chromosome arm” or like terms refers to the coefficient of variation (CV) of the telomere lengths for a specific chromosome arm in a group of cells. Telomere length variation at a specific chromosome arm is thus a measure of the variability of telomere lengths of a specific chromosome arm in a group of cells. “Reference telomere length variation at a specific chromosome arm” or like terms refers to a telomere length variation established for a specific chromosome arm from a sample(s) from a subject(s) that is considered a control. A reference telomere length variation at a specific chromosome arm could be, for example, from healthy individuals or from non-cancerous patients. It is understood that the reference telomere length variation at a specific chromosome arm can be produced de novo or can be a number previously determined as a reference length.

“Within-Cell Telomere Length Variation (WCTLV) or like terms refers to the coefficient of variation (CV) of the telomere lengths among all of the non-homologous chromosome arms in a cell, where the telomere lengths of both homologous telomeres of a chromosome are combined. WCTLV is thus a measure of the variability of telomere lengths in a cell. A typical human cell has 46 non-homologous chromosome arms. “Reference WCTLV” or like terms refers to a WCTLV established from a sample(s) from a subjects) that is considered a control. A reference WCTLV could be, for example, from healthy individuals or from non-cancerous patients. It is understood that the reference WCTLV can be produced de novo or can be a number previously determined as a reference length.

Within-Cell Telomere Length Variation (WCTLV) can use absolute telomere length or relative telomere length. For example, WCTLV using absolute telomere length can be the coefficient of variation (CV) of the absolute telomere lengths among all of the non-homologous chromosome arms in a cell, where the telomere lengths of both homologous telomeres of a chromosome are combined. WCTLV using absolute telomere length can be referred to as AWCTLV. WCTLV using relative telomere length can be the coefficient of variation (CV) of the relative telomere lengths among all of the non-homologous chromosome arms in a cell, where the telomere lengths of both homologous telomeres of a chromosome are combined. WCTLV using relative telomere length can be referred to as RWCTLV.

“Within-Cell Homolog Telomere Length Variation (WCHTLV) or like terms refers to the coefficient of variation (CV) of the relative telomere lengths among all of the chromosome arms in a cell. WCHTLV is thus a measure of the variability of telomere lengths in a cell.

“Within-Set Telomere Health Variation (WSTHV) or like terms refers to the coefficient of variation (CV) of the relative telomere healths among a set of non-homologous chromosome arms, where the telomere healths of both homologous telomeres of a chromosome are combined. WSTHV is thus a measure of the variability of telomere healths in a set of chromosomes.

“Within-Set Telomere Length Variation (WSTLV) or like terms refers to the coefficient of variation (CV) of the relative telomere lengths among a set of non-homologous chromosome arms, where the telomere lengths of both homologous telomeres of a chromosome are combined. WSTLV is thus a measure of the variability of telomere lengths in a set of chromosomes.

“Within-Set Homolog Telomere Length Variation (WSHTLV) or like terms refers to the coefficient of variation (CV) of the relative telomere lengths among a set of chromosome arms. WSHTLV is thus a measure of the variability of telomere lengths in a set of chromosome arms.

“CV (coefficient of variation)” or like terms is the percent of the standard deviation of a group of measurements divided by the average value of this group of measurements. For example, if the standard deviation of a group telomere lengths is 9600 and the average length of this group of telomeres is 19200, then the CV of these telomeres is 9600/19200*100=50%.

“Frequency of extremely short or long telomeres” or like terms is number of extremely short telomeres or long telomeres divided by the total number of telomeres measured.

“Extremely short telomere” or like terms is a telomere that has the length that is shorter than 25% of the average length of a telomere for a person. Sometimes, extremely short telomere can be defined as a telomere that has the length that is shorter than 10% of the average length of a telomere for a person. Sometimes, extremely short telomere can be defined as a telomere that has the length that is shorter than 1% of the average length of a telomere for a person.

“Extremely long telomere” or like terms is a telomere that has the length that is longer than 200% of the average length of a telomere for a person. Sometimes, extremely long telomere can be defined as a telomere that has the length that is longer than 300% of the average length of a telomere for a person. Sometimes, extremely long telomere can be defined as a telomere that has the length that is longer than 400% of the average length of a telomere for a person.

“Chromosome 15 telomere(s)” or like terms refers to the telomere(s) of chromosome 15. Similar terms can be used for any chromosome arm by substituting the chromosome number and arm designation. For example, chromosome 9 telomere and chromosome X telomere refer to the telomere of chromosome 9 and of chromosome X, respectively.

“Chromosome 15p telomere” or like terms refers to the telomere on the short arm of chromosome 15. Similar terms can be used for any chromosome arm by substituting the chromosome number and arm designation. For example, chromosome 9p telomere, chromosome Xp telomere, and chromosome 15q telomere refer to the telomere on the short arm of chromosome 9, the short arm of chromosome X, and the long arm of chromosome 15, respectively.

“Chromosome 15p-S telomere” or like terms refers to the shorter telomere of the homologous pair of telomeres of the short arm of chromosome 15. “Chromosome 15p-L telomere” or like terms refers to the longer telomere of the homologous pair of telomeres of the short arm of chromosome 15. Similar terms can be used for any chromosome arm by substituting the chromosome number and arm designation. For example, chromosome 9p-S telomere, chromosome Xp-S telomere, and chromosome 15q-S telomere refer to the shorter telomere of the homologous pair of telomeres on the short arm of chromosome 9, the shorter telomere of the homologous pair of telomeres on the short arm of chromosome X, and the shorter telomere of the homologous pair of telomeres on the long arm of chromosome 15, respectively.

“Chromosome 15 telomere length” or like terms refers to the length of the telomeres of chromosome 15. Similar terms can be used for any chromosome arm by substituting the chromosome number and arm designation. For example, chromosome 9 telomere length and chromosome X telomere length refer to the telomere length of chromosome 9 and of chromosome X, respectively.

“Chromosome 15p telomere length” or like terms refers to the length of the telomere of the short arm of chromosome 15. Similar terms can be used for any chromosome arm by substituting the chromosome number and arm designation. For example, chromosome 9p telomere length, chromosome Xp telomere length, and chromosome 15q telomere length refer to the telomere length of the short arm of chromosome 9, the short arm of chromosome X, and the long arm of chromosome 15, respectively.

“Chromosome 15p-S telomere length” or like terms refers to the length of the shorter telomere of the homologous pair of telomeres of the short arm of chromosome 15. “Chromosome 15p-L telomere length” or like terms refers to the length of the longer telomere of the homologous pair of telomeres of the short arm of chromosome 15. Similar terms can be used for any chromosome arm by substituting the chromosome number and arm designation. For example, chromosome 9p-S telomere length, chromosome Xp-S telomere length, and chromosome 15q-S telomere length refer to the length of the shorter telomere of the homologous pair of telomeres on the short arm of chromosome 9, the shorter telomere of the homologous pair of telomeres on the short arm of chromosome X, and the shorter telomere of the homologous pair of telomeres on the long arm of chromosome 15, respectively.

“Chromosome 15p reference length” or like terms refers to a reference length of the short arm of chromosome 15. Similar terms can be used for any chromosome arm by substituting the chromosome number and arm designation.

“Chromosome 15 telomere ratio” or like terms refer to a telomere ratio where the numerator of the telomere ratio is represented by the direct or indirect length of chromosome 15 telomeres. Similar terms can be used for any chromosome arm by substituting the chromosome number and arm designation.

“Chromosome 15p telomere ratio” or like terms refer to a telomere ratio where the numerator of the telomere ratio is represented by the direct or indirect length of chromosome 15p telomeres. Similar terms can be used for any chromosome arm by substituting the chromosome number and arm designation.

“Chromosome 15p-S telomere ratio” or like terms refer to a telomere ratio where the numerator of the telomere ratio is represented by the direct or indirect length of the shorter telomere of the homologous pair of telomeres of the short arm of chromosome 15. “Chromosome 15p-L telomere ratio” or like terms refer to a telomere ratio where the numerator of the telomere ratio is represented by the direct or indirect length of the longer telomere of the homologous pair of telomeres of the short arm of chromosome 15. Similar terms can be used for any chromosome arm by substituting the chromosome number and arm designation.

“Risk” or like terms refers to the chance, probability, likelihood, etc. that an event, characteristic, condition, etc. will be present or will occur. Risk can be expressed, for example, numerically, quantitatively, qualitatively, as a ratio, as a percentage, or in other appropriate ways. Risk can be expressed with or without reference to other subjects, states, averages, means, medians, and/or quartile values of the reference values.

“Increased likelihood of having cancer” or “increased likelihood of contracting cancer” or like terms refers to an odds ratio as discussed herein where the condition is cancer. For example, it can be considered a fold likelihood relative to a group, such as a subject has at least a 3.0, 3.9, or 6.6, fold increase relative to all women, or at least a 2.1, 2.9, or 6.2, fold increase relative to all premenopausal women or at least a 4.3, 5.1, or 7.5, fold increase relative to all postmenopausal women.

“Odds ratio” (OR) or like terms refers to a ratio of the risk or odds that a subject or a group will have a characteristic or condition relative to another group. For example, a subject can be said to have an odds ratio of 3.0 relative to a group when the subject has a 3.0 fold greater risk or odds of having the characteristic or condition relative to the odds or risk of a subject in the group having the characteristic or condition.

“Adjusted odds ratio” (aOR) or like terms refers to an odds ratio that has taken into account other related factors, such as statistically adjusted, based on one or more characteristics (such as demographic or lifestyle characteristics) obtained in a subject history from the subject the telomere ratio was obtained from.

“Measuring the length of the chromosome telomere” or “measuring telomere length” or “quantitating telomere length” or like terms refers to determining the length of a chromosome telomere by any means, including by directly measuring, such as by determining the number of bases or repeats or other physical ways of quantifying the absolute length of a telomere, as well as by indirectly measuring the length. An indirect measurement of the length refers to measuring something that is a substitute or related or correlated to length, such as the amount of a telomere marker bound to a telomere, or the amount of signal arising from a telomere marker bound to a telomere, such as the amount of fluorescence bound to a telomere via a telomere marker.

Telomere length can be measured by, for example, quantifying the fluorescence using TeloMeter, which is a program that is freely available from John Hopkins University website (internet site bui2.win.ad.jhu.edu/telometer/) or quantitating can arise from the commercially available Isis image software from Metasystems (website www.metasystems.com/).

Telomere length can be expressed in any suitable form. For example, telomere length can be expressed as a number of nucleotides, as a number of telomere sequence repeats, as a length by reference to a standard such as the meter, as a signal or value form an indirect measurement, etc.

It is understood that a single telomere or arm of a telomere can be measured, multiple telomeres can be measured, a specific number or set of telomeres can be measured, etc., up to and including all of the telomeres of each chromosome of a cell of a subject. There are 23 pairs of chromosomes, 46 individual chromosomes, 92 chromosomal arms, and 92 telomeres in a typical human cell.

“Indirect measurement” or like terms refer to a measurement that is representative of something else. For example, the amount of fluorescence signal arising from bound fluorescently labeled probe on a telomere of a chromosome arm is an indirect measurement of the length or the telomere of that chromosome arm.

“Reference length” or like terms refers to a length established from a sample(s) from a subjects) that is considered a control. A reference length could be, for example, from healthy individuals or from non-cancerous patients. It is understood that the reference length can be produced de novo or can be a number previously determined as a reference length. For example, reference chromosome telomere length can be the average chromosome telomere length in cells from normal subjects of similar type to the cell being assessed, reference chromosome telomere length can be the average chromosome telomere length of the chromosome being assessed in cells from normal subjects of similar type to the cell being assessed, and reference chromosome telomere length can be the average arm-specific telomere length in cells from normal subjects of similar type to the cell being assessed. As another example, reference chromosome telomere length can be the median of the chromosome telomere lengths in cells from normal subjects of similar type to the cell being assessed, reference chromosome telomere length can be the median of the chromosome telomere lengths of the chromosome being assessed in cells from normal subjects of similar type to the cell being assessed, and reference chromosome telomere length can be the median of the arm-specific telomere lengths in cells from normal subjects of similar type to the cell being assessed. As another example, reference chromosome telomere length can be the quartile value of the chromosome telomere lengths in cells from normal subjects of similar type to the cell being assessed, reference chromosome telomere length can be the quartile value of the chromosome telomere lengths of the chromosome being assessed in cells from normal subjects of similar type to the cell being assessed, and reference chromosome telomere length can be the quartile value of the arm-specific telomere lengths in cells from normal subjects of similar type to the cell being assessed. Generally, by “cells . . . of similar type to the cell” is meant that the first cells are the same type of cells as the second cells, similar to the type of the second cells, where “type of cells” can refer to the cell type of the cells, the tissue type of the cells, the organ from which the cells come, or a combination. As disclosed herein in the context of telomeres, a reference length could be 100, 200, 500, 1000, 10000, 100000, 1000000, 5000000, in fluorescent intensity units (FIU). As disclosed herein, a reference relative telomere length could be 0.00494, 0.00583, or 0.00680 or other like numbers, disclosed in tables 1-4, for example.

“Reference telomere parameters” or like terms refers to a telomere parameters that are produced from a sample(s) from a subject(s) that is considered a control. For example, the sample(s) could be from healthy individuals or from non-cancerous patients. It is understood that the reference telomere parameters can be produced de novo or can be a number previously determined as a reference number.

“Telomere marker” or like terms refers to any molecule or substance that interacts preferentially with a telomere relative to another region of a chromosome. A telomere marker could be, for example, a hybridization probe for a telomere, such as fluorescent labeled telomere sequences of certain length, such as 18 base pairs.

“Shorter” or “shorter length” or like terms refers to, in the context of nucleic acids, chromosomes, telomeres, etc., fewer nucleotides. One nucleic acid, such as a chromosome or a telomere of a chromosome, would be shorter than another nucleic acid if it has at least one fewer nucleotide. “Detectably shorter” or like terms refers to, in the context of nucleic acids, chromosomes, telomeres, etc., detectably fewer nucleotides. Detectably shorter generally is in the context of the manner in which length is measured since different ways of measuring can have different thresholds of detectability.

How much shorter a nucleic acid is than another nucleic acid can be represented by, for example, referring to the representative length of the two nucleic acids as less than or equal to 0.000001, 0.00001, 0.0001, 0.001, 0.01, 0.1, 0.2, 0.3, 0.4, 0.5, 0.6, 0.7, 0.8, 0.9, or 0.95 of the length of one or the other nucleic acid. For example, a nucleic acid that is 900 bases long is 0.9 the length of a nucleic acid that is 1000 bases long.

“Longer” or “longer length” or like terms refers to, in the context of nucleic acids, chromosomes, telomeres, etc., more nucleotides. One nucleic acid, such as a chromosome or a telomere of a chromosome, would be longer than another nucleic acid if it has at least one more nucleotide. “Detectably longer” or like terms refers to, in the context of nucleic acids, chromosomes, telomeres, etc., detectably more nucleotides. Detectably longer generally is in the context of the manner in which length is measured since different ways of measuring can have different thresholds of detectability.

How much longer a nucleic acid is than another nucleic acid can be represented by, for example, referring to the representative length of the two nucleic acids as greater than or equal to 100,000, 10,000, 1,000, 100, 10, 9, 8, 7, 6, 5, 4, 3, 2, 1.9, 1.8, 1.7, 1.6, 1.5, 1.4, 1.3, 1.2, 1.1, 1.09, 1.08, 1.07, 1.06, 1.05, 1.04, 1.03, 1.02, 1.01 of the length of one or the other nucleic acid. For example, a nucleic acid that is 1100 bases long is 1.1 the length of a nucleic acid that is 1000 bases long.

“Greater” or like terms refers to more of something than in a comparison value, composition, component, etc. Various measures can be greater than other measures. For example, telomere health, length, such as telomere length, variation, such as variation of telomere length, average, mean, median, quartile value, deviation, standard deviation, etc. can be measures that are greater than comparison measures. Telomere health would be greater than a comparison telomere health if a measure of telomere health is greater for the telomere than for the measure of telomere health for the comparison telomere. Variation in telomere length would be greater than a comparison variation in telomere length if a measure of variation is greater for the telomere length than for the measure of variation for the comparison telomere length.

“Less” or like terms refers to less or fewer of something than in a comparison value, composition, component, etc. Various measures can be less than other measures. For example, telomere health, length, such as telomere length, variation, such as variation of telomere length, average, mean, median, quartile value, deviation, standard deviation, etc. can be measures that are less than comparison measures. Telomere health would be less than a comparison telomere health if a measure of telomere health is less for the telomere than for the measure of telomere health for the comparison telomere. Variation in telomere length would be less than a comparison variation in telomere length if a measure of variation is less for the telomere length than for the measure of variation for the comparison telomere length.

“Higher variation” or like terms refers to higher variability in length among a group of measured telomeres. For example, in a group of measured telomeres, if some telomeres are very long and some are very short, then, telomere length variation will be high. In contrast, if all measured telomeres have similar length, then telomere length variation will be low.

How much higher the telomere variation of one group of telomeres is than the telomere variation of another group of telomeres can be represented by referring to the representative measures of two groups of telomeres as less than or equal to 0.1%, 1%, 10%, 20%, 30%, 40%, 50%, 60%, 70%. 80%, 90%, 100%, 200%, 300%, 400%, 500%, 600%, 700%, 800%, 900%, 1000% of the CV of a group or the other group of the telomeres. For example, a group of telomeres that have 60% CV is 10% higher in telomere length variation than a group of telomeres that have 50% CV.

“Subject” or like terms refers to an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

“Sample” or like terms refers to an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, serum, plasma, lymphatic fluid, mucus, urine, stool, saliva, tears, bile) that contains cells or cell components.

“Cancer” or “cancerous” or like terms refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include breast cancer, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

“Treatment” or “treating” or like terms refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount.

“Patient history” or “subject history” or like terms refers to one or more items in the history of a subject which could be considered relevant to the subject, such as race, age, gender, physical status, such as pre- or post-menopausal, pregnant, diabetic, overweight or obese, smoking, alcohol consumption, family cancer history, or like items.

“Preventive treatment” or “preventative intervention” or like terms for cancer refer to current or future preventive treatment regimes or protocols that are designed to reduce the likelihood of a subject getting a disease or condition. In the context of cancer, preventative treatments include current or future preventive treatment regimes or protocols that are designed to reduce the likelihood of a subject getting cancer. Current preventative treatment regimes are those in use by physicians or health care organization. For example, Tamoxifen is prescribed for women who are at high risk of breast cancer or bilateral surgically removal of ovaries are used to reduce the breast or ovarian cancer if a women is at very high risk of breast cancer, i.e., BRCA1 mutation carriers. Preventive treatment/intervention for cancer also refers to clinical protocols to monitor an individual who is at high risk of getting cancer more closely to detect a cancer early. Patients whose cancer is detected in a early stage usually has a better change of cure and survival.

“Fresh whole blood” refers to a sample of venous blood from a subject and was kept at 4° C. for less than 48 hours.

“Fluorescent” or like terms refers to luminescence that is caused by the absorption of radiation at one wavelength followed by nearly immediate reradiation usually at a different wavelength and that ceases almost at once when the incident radiation stops, as understood in the art.

“Mimic” or like terms refers to performing one or more of the functions of a reference object. For example, a molecule mimic performs one or more of the functions of a molecule.

“Obtaining” or like terms refers to getting or acquiring. For example, obtaining a sample includes taking a sample physically from a subject and it also includes receiving a sample which someone else took from a subject, which was for example, stored. Thus, obtaining includes but is not limited to physically collecting a sample.

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

The singular forms “a,” “an” and “the” or like terms include plural referents unless the context clearly dictates otherwise. Thus, for example, reference to “a pharmaceutical carrier” includes mixtures of two or more such carriers, and the like.

Abbreviations, which are well known to one of ordinary skill in the art, may be used; for example, “h” or “hr” for hour or hours, “g” or “gm” for gram(s), “mL” for milliliters, and “rt” for room temperature, “nm” for nanometers, “M” for molar, and like abbreviations.

“About” modifying, for example, length, ratio, the quantity of an ingredient in a composition, concentrations, volumes, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

The word “or” or like terms means any one member of a particular list and also includes any combination of members of that list.

Specific and preferred values disclosed for lengths, ratios, components, compounds, levels, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Ranges can be expressed herein as from “about” one particular value, and/or to “about” another particular value. When such a range is expressed, other forms include from the one particular value and/or to the other particular value. Similarly, when values are expressed as approximations, by use of the antecedent “about,” it will be understood that the particular value forms another form. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Comprise” and variations of the word, such as “comprising” and “comprises,” means “including but not limited to,” and is not intended to exclude, for example, other additives, components, integers or steps.

“Consisting essentially of” refers to, for example, the stated subject matter plus other components or steps that do not materially affect the basic and novel properties of the stated subject matter.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

The disclosed methods, compositions, articles, and machines, can be combined in a manner to comprise, consist of, or consist essentially of, the various components, steps, molecules, and composition, and the like, discussed herein.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Compounds and compositions have their standard meaning in the art. It is understood that wherever a particular designation, such as a molecule, substance, marker, cell, or reagent is disclosed, compositions comprising, consisting of, and consisting essentially of these designations are also disclosed.

Unless defined otherwise, all technical and scientific terms used herein have the same meanings as commonly understood by one of skill in the art to which the disclosed method and compositions belong. It is understood that the disclosed method and compositions are not limited to the particular methodology, protocols, and reagents described as these may vary. Although any methods and materials similar or equivalent to those described herein can be used in the practice or testing of the present method and compositions, the particularly useful methods, devices, and materials are as described.

Those skilled in the art will recognize, or be able to ascertain using no more than routine experimentation, many equivalents to the specific embodiments of the method and compositions described herein. Such equivalents are intended to be encompassed by the following claims.

### II. Telomeres

Telomeres are specialized DNA-protein structures that cap the ends of linear chromosomes. They are crucial for protecting linear chromosomes and are essential for maintaining the integrity and stability of genomes (McEachern et al. Annu. Rev Genet, 34:331-358, 2000). Telomere-induced chromosomal instability could drive the tumorigenic process by increasing mutation rates for oncogenes and tumor suppressor genes (Maser et al. Science, 297:565-569, 2000).

Disclosed herein, a case-control study of breast cancer, examining the association between chromosome arm-specific telomere lengths and breast cancer risk, demonstrated that short telomere lengths on specific chromosomal arms, such as 9p, Xp, and 15p, are strongly associated with breast cancer risk, and provide new methods and compositions for breast cancer risk assessment for individuals in the general population.

Disclosed herein, a case-control study of breast cancer, examining the association between homolog telomere length difference (HTLD) and breast cancer risk, demonstrated that higher HTLD on specific chromosomal arms, such as 5q, Xp, 9p, 12p, 15p and 15q, are strongly associated with breast cancer risk in pre-menopausal women, and provide new methods and compositions for breast cancer risk assessment for premenopausal women.

Disclosed herein, a case-control study of breast cancer, examining the association between overall telomere length variation and breast cancer risk, demonstrated that higher telomere length variation are significantly associated with breast cancer risk in pre-menopausal women, and provide new methods and compositions for breast cancer risk assessment for premenopausal women.

### III. Samples

The disclosed methods use samples that include cells in order to assess telomere lengths. Useful samples include body fluids or excretions. Such samples, especially blood-based samples, are usually easier or more convenient to obtain and contain cells the telomeres of which have been discovered to be relevant for assessing cancer risk. Numerous methods and techniques are known for obtaining, preparing, storing, and using biological and cell samples, including especially blood-based samples, and such methods and techniques can be used with the disclosed methods. Sample can be obtained by taking a sample physically from a subject or receiving a sample which someone else took from a subject and which was, for example, stored. Useful samples include, for example, blood, serum, plasma, lymphatic fluid, mucus, urine, stool, saliva, tears, bile that contains cells or cell components, preferable fresh whole blood.

Samples should be obtained from subjects in which cancer risk is to be assessed. Human subjects are most preferred for the disclosed methods, but samples can also be obtained from other subjects.

### IV. Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

### V. Nucleic Acids

There are a variety of molecules disclosed herein that are nucleic acid based, as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

A. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate complementary nucleic acid sequences.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

B. Sequences

There are a variety of sequences related to telomeres disclosed herein that are disclosed on Genbank, and these sequences and others are herein incorporated by reference in their entireties as well as for individual subsequences contained therein.

A variety of sequences are provided herein and these and others can be found in Genbank, at webpage www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

### VI. Cancer

A. Breast Cancer

Breast cancer, like most human malignancies, is characterized by short or extremely long telomere in tumor cells and chromosomal instability (Baudis BMC Cancer 7:226, 2007; Shih et al. Cancer Res 61:818-822, 2001; Michor et al. Semin Cancer Biol 15:43-49, 2005). It is documented that chromosomal instability preferentially involves specific chromosome arms for each type of human cancer (Baudis BMC Cancer 7:226, 2007). In breast cancer, frequent chromosomal abnormalities in early stage breast tumors involves a few chromosomal arms, including gains of 1q, 8q, 17q, and 20q, and losses of 8p, 9p, 16q and 17p (Baudis BMC Cancer 7:226, 2007; Gorgoulis et al. Mol Med 4:807-822, 1998; An et al. Genes Chromosomes Cancer 17:14-20, 1996). Disclosed herein, chromosome arm-specific telomere deficiency was correlated with the cancer which is consistent with the underlying mechanisms for such arm-specific instability because critically short/dysfunctional telomeres can lead to chromosome end fusion which induces chromosome specific instability via repeated series of breakage-fusion-bridge (BFB) cycles (Hackett et al. Cell, 106:275-286, 2001; Gisselsson et al. PNAS 98:12683-12688, 2001; Stewenius et al. PNAS, 102:5541-5546, 2005; Lo et al. Neoplasia, 4:531-538, 2002).

Breast cancer, like most human malignancies, is characterized by chromosomal instability (CIN) (Baudis BMC Cancer 7:226, 2007). CIN is featured by losses or gains of entire chromosomes or chromosomal fragments, resulting in aneuploidy, large deletions or gains, and chromosomal rearrangements. CIN is observed as an early event in tumorigenesis (Shih et al. Cancer Res 61:818-822, 2001; Michor et al. Semin Cancer Biol 15:43-49, 2005) and there is abundant evidence of correlation between increasing chromosomal abnormalities and greater tumor aggressiveness. However, the molecular defects underlying CIN, and whether CIN is a cause or a consequence of the malignant phenotype, are not clear.

Accumulating evidence indicates that dysregulation of the p53 and Rb pathways are important mechanisms of CIN development (Hernando et al. Nature 430:797-802, 2004), and deregulation of the Rb pathway is a major contributor to CIN in breast tumors (Fridlyand et al. BMC Cancer 6:96, 2006; Gorgoulis et al. 4:807-822, 1998). One of the chromosomal abnormalities that affects the regulation of both p53 and Rb pathways is the chromosome 9p21 deletion. Indeed, deletions of chromosome 9 or 9p21 are the most frequent early chromosomal abnormalities in cancers, including head and neck (Miracca et al. 9:229-233, 2000), bladder (Mhawech-Fauceglia et al. Cancer 106:1205-1216, 2006), non-small cell lung (Sato et al. Genes Chromosomes Cancer 44:405-414, 2005) and skin cancers (Baudis et al. 2007; Rakosky et al. Cancer Genet Cytogenet 182:116-121, 2008). Frequent chromosome 9p deletions were also reported for high grade ductal carcinoma in situ (DCIS) (Ellsworth et al. Ann Surg Oncol 14:3070-3077, 2007; Hwang et al. Clin Cancer Res 10:5160-5167, 2004) and invasive breast cancer (An et al. Genes Chromosomes Cancer 17:14-20, 1996; Xie et al. Int J Oncol 21:499-507, 2002). On 9p, deletions and recombination are centered around an important tumor suppressor locus, the CDKN2A (Williamson et al. Hum Mol Genet 4:1569-1577, 1995; Cairns et al. Nat Genet 11:210-212, 1995). The CDKN2A gene encodes 2 proteins (p161NK4 and p14ARF) that regulate two critical cell cycle regulatory pathways: the p53 pathway and the retinoblastoma pathway (Harris et al. Oncogene 24:2899-2908, 2005). Thus inactivation of the CDKN2A locus via chromosome 9p21 deletion may be an initiating event in the development of breast cancer. Chromosomes possessing short telomeres may be unstable and so individuals who have short telomeres on chromosome 9 may have an increased likelihood of chromosome 9 deletion, and consequently a greater risk to develop cancer.

### VII. Methods for Breast and Lung Cancer Risk Assessment

One aspect of the present disclosure is directed to methods for detecting altered telomere parameters as diagnostic indicators of cancer risk, such as breast cancer and lung cancer. More particularly, in accordance with one embodiment methods are provided for detecting the presence of telomere shortening in the chromosomes prepared from blood cells of a subject. Being able to perform diagnostic tests for cancer risk, such as breast cancer and lung cancer, from blood cells, such as lymphocyte cells of the patient, is desirable in the field of cancer risk assessment. Disclosed is data showing that telomere lengths, such as chromosome 9p, Xp, 15p lengths, such as lengths of the short arms of chromosomes 9, 15, and X, have been associated with the risk of cancer and can serve as possible markers for cancer risk prediction. Also disclosed is data showing that variation in telomere length and the frequency of extremely short telomeres, which indicate telomere health, are strongly associated with lung cancer risk.

Disclosed are methods of calculating telomere length variation as additional parameters to measure an individual's telomere health, using telomere length measured from blood lymphocytes. Disclosed is the data showing that high variation in length on chromosomes 5q, Xp, 9p, 12p, 15p and 15q, such as the chromosomes 5q, Xp, 9p, 12p, 15p and 15q HTLD, have been associated with the risk of cancer and can serve as possible markers for cancer risk prediction.

Disclosed is data showing that high variation in length among the 92 telomeres in a typical human cell, such as the CV of 92 telomeres in a typical human cell, have been associated with the risk of cancer and can serve as possible markers for cancer risk prediction.

Disclosed are methods of detecting the presence of shortened telomeres in blood cells is provided using a chromosome analysis based on quantitating telomere length disclosed herein. The method comprises the steps of obtaining blood from a subject, harvesting the chromosomes, performing telomere fluorescent in situ hybridization on chromosomes, quantitating telomere length and determining the cancer risk based on the length of a specific chromosome arm, such as Xp telomere or on the length variation of a specific chromosome arm, such as 9p telomere, or on the length variation of 92 telomeres of a typical human cell.

In one embodiment, the chromosome analysis is performed using telomere fluorescent in situ hybridization (FISH).

A significant advantage of the disclosed methods is that blood cells are used instead of tissue derived directly from breast, lung, or tumor. Therefore the test is much less invasive and can be used as pre-cancer screen as opposed to a post-cancer screen.

It was discovered that telomere health is strongly associated with breast cancer risk and lung cancer risk. For example, individual telomere length and variation in telomere length, which indicate telomere health, are strongly associated with breast cancer risk. Specific examples include significant association of breast cancer risk in premenopausal women with measures of telomere health such as short telomere length on chromosomes Xp and 15p, greater length differences between homologous telomeres on chromosomes 9p, 15p and 15q, greater telomere length variation in lymphocytes on chromosome 18p, and greater variation in telomere length among the chromosomes of a cell are.

The data herein provide the first evidence that telomere health or deficiency on certain chromosome arms are linked to breast cancer susceptibility and risk. These new discoveries have clinical application in detecting and assessing cancer risk, the application of which is the subject of the disclosed methods. The disclosed telomere-related parameters can be used, for example, as a panel of blood-based biomarkers for cancer risk detection and assessment. The disclosed telomere health measures such as chromosome telomere length measurements and assessments can be incorporated into the current and future prediction and prognosis models to enhance breast cancer risk prediction and prognosis. The disclosed methods can be used to improve the efficiency of, for example, both population-based preventive programs, such as screening mammography and chest x-rays, and individual-based preventive strategies such as chemoprevention by targeting women who are at the greatest risk for breast cancer.

The results of the experiments discussed in the Examples, show that, after adjustment for known breast cancer risk factors, higher homologous telomere length difference and shorter telomere length on chromosome 9p was strongly associated with an increased risk of breast cancer, as described at least in Tables 1-4. This finding indicates that individuals who possess poor telomere health on short arm of chromosome 9 are at increased risk of breast cancer.

The results in the Examples also show that pre-menopausal women have an increased risk of breast cancer if the chromosome telomere length of chromosome telomere 1p-S, Xp-S, 9p-S, or 15p-S is less than the reference chromosome telomere length. The data also show that post-menopausal women have an increased risk of breast cancer if the chromosome telomere length of chromosome telomere 15p-S is less than the reference chromosome telomere length. The data also show that pre-menopausal women have greater risk of breast cancer the shorter the chromosome telomere length of chromosome telomere Xp-S or 15p-S is less than the reference chromosome telomere length.

Differences in the parameters of chromosome telomere health of homologous telomeres (telomeres in homologous chromosome arms) can also be used to assess a subject's risk of cancer. For example, the results in the Examples show that pre-menopausal women have an increased risk of breast cancer if the homologous telomere length difference (HTLD) is higher in chromosome arm 5q, Xp, 8q, 9p, 12p, 15p, or 15q than a reference HTLD, such as the HTLD for the chromosome arm in normal subjects. The data also show that pre-menopausal women have a greater risk of breast cancer the higher the HTLD in chromosome arm Xp, 9p, 15p, or 15q.

The level of variability in telomere health within a cell (such as between the telomeres of a cell) can also be used to assess cancer risk. For example, pre-menopausal women have an increased risk of breast cancer if the within-cell telomere length variation (WCTLV) is higher than a reference WCTLV, such as the WCTLV in normal subjects. As another example, pre-menopausal female have greater risk of breast cancer the higher the WCTLV.

In humans, there are 23 pairs of chromosomes and 92 telomeres, and chromosome specific telomere lengths are highly polymorphic between chromosomal arms (Lansdorp et al. Hum Mol Genet 5:685-691, 1996; Graakjaer et al. Mech Ageing Dev 124:629-640, 2003; Martens et al. Nat Genet 18:76-80, 1998). The disclosed observation that the poor chromosome 9p telomere health, such as short 9p telomere and greater HTLD on 9p being strongly associated with breast cancer risk, provides methods of using this information to assess the risk of cancer, such as breast cancer, in a subject. This study reports that a short telomere on chromosome 9p is strongly associated with breast cancer risk. Telomere length on chromosome 9p, as disclosed herein, is a tool for identifying women at risk for breast cancer and improving breast cancer risk assessment for individuals in general population.

Telomere lengths on chromosome 9q were not associated with breast cancer risk. No significant correlations was observed between 9p and 9q telomere lengths in controls, except a weak correlation between 9p-short and 9q-short, suggesting telomere lengths on 9p or 9q are independent events. There was no significant difference in mean overall (cell total) telomere length between cases and controls (Table 1) (Zheng et al. Breast Cancer Res Treat, 2009). This may in part explain the null findings by three recent studies that examined the association of overall (cell total) telomere length in blood leucocytes and breast cancer risk (Shen et al. Cancer Res 67:5538-5544, 2007; Svenson et al. Cancer Res 68:3618-3623, 2008; Barwell et al. Br J Cancer 97:1696-1700, 2007).

Disclosed are methods of assaying a subject comprising, Obtaining a sample from the subject, Measuring the length of each 92 telomeres in a typical human cell, such as chromosome 9p or 15p telomeres, producing a mathematical calculation of 188 parameters that defines overall or chromosome arm specific telomere health status, such as chromosome 9p telomere health, and Identifying a subject having poor telomere health overall or on specific chromosome arm which is poor than a telomere health in a reference group of healthy people. Telomere parameters can be used alone or in any combination to determine telomere health status for an individual.

Also disclosed are methods, wherein shorter comprises chromosome 9p telomere length less than or equal to 0.000001, 0.00001, 0.0001, 0.001, 0.01, 0.1, 0.2, 0.3, 0.4, 0.5, 0.6, 0.7, 0.8, 0.9, or 0.95 of the chromosome 9p reference length or alone or in any combination.

Disclosed are methods, wherein a subject having a telomere ratio less than a reference telomere ratio has an increased likelihood of having cancer, wherein a subject having a telomere ratio less than a reference telomere ratio has an increased likelihood of contracting cancer, wherein the likelihood indicates at least a 3.0, 3.9, or 6.6, fold increase relative to all women, wherein the likelihood indicates at least a 2.1, 2.9, or 6.2, fold increase relative to all premenopausal women, wherein the likelihood indicates at least a 4.3, 5.1, or 7.5, fold increase relative to all postmenopausal women, wherein the cancer is breast cancer, wherein the telomere ratio is a chromosome 9 telomere ratio, wherein the telomere ratio is a chromosome 9p telomere ratio, wherein the reference telomere ratio is 0.00494, 0.00583, or 0.00680, wherein measuring the telomere ratio comprises substituting an indirect measurement of telomere length represented in the telomere ratio, wherein measuring the telomere ratio comprises substituting an indirect measurement of telomere length for all telomeres represented in the telomere ratio, wherein indirect measurement comprises measuring a telomere marker, wherein the telomere marker comprises a telomere hybridization probe, wherein the hybridization probe comprises a fluorescent probe, wherein the indirect measurement comprises the fluorescent signal of a fluorescent in situ hybridization assay, wherein measuring the telomere ratio comprises the length of at least one telomere represented in the telomere ratio, wherein measuring the telomere ratio comprises the length of all telomeres represented in the telomere ratio, further comprising the step of measuring the length of all of the telomeres in the cell of the subject, producing a total telomere length, further comprising the step of comparing the length of the chromosome 9p in the cell of the subject to the length of all of the telomeres in the cell of the subject forming a telomere ratio, and/or further comprising the step of creating a telomere ratio, alone or in any combination.

Disclosed are methods of assaying a subject comprising measuring parameters of the health of at least one chromosome telomere of a chromosome in at least one cell of a sample from a subject, thereby producing a chromosome telomere health for at least one of the chromosome telomeres, and comparing the chromosome telomere health with a reference chromosome telomere health. By measuring parameters of the health of individual chromosome telomeres and comparing to parameters of reference chromosome telomere healths, cancer risk in subjects can be assessed. Any or a combination of parameters of chromosome telomere health can be used for such measurements. In some forms, the parameter of the chromosome telomere health can be the length of the chromosome telomere. In some forms, the parameter of the reference chromosome telomere health can be a reference chromosome telomere length.

For example, pre-menopausal women have an increased risk of breast cancer if the chromosome telomere health of chromosome telomere 1p-S, Xp-S, 9p-S, or 15p-S is less than the reference chromosome telomere health. As another example, post-menopausal women have an increased risk of breast cancer if the chromosome telomere health of chromosome telomere 15p-S is less than the reference chromosome telomere health. As another example, pre-menopausal women have greater risk of breast cancer the shorter the chromosome telomere health of chromosome telomere Xp-S or 15p-S is less than the reference chromosome telomere health.

Reduced chromosome telomere health can be established using any parameter or degree of chromosome telomere health less than the reference chromosome telomere health. For example, the chromosome telomere health can be a fraction of the reference chromosome telomere health. For example, in the case where the parameter of chromosome telomere health is chromosome telomere length, the chromosome telomere length can be less than or equal to 0.5 of the reference chromosome telomere length (as the appropriate reference chromosome telomere health).

Differences in the parameters of chromosome telomere health of homologous telomeres (telomeres in homologous chromosome arms) can also be used to assess a subject's risk of cancer. For example, pre-menopausal women have an increased risk of breast cancer if the homologous telomere length difference (HTLD) is less in chromosome arm 5q, Xp, 8q, 9p, 12p, 15p, or 15q than a reference HTLD, such as the HTLD for the chromosome arm in normal subjects. As another example, pre-menopausal women have a greater risk of breast cancer the lower the HTLD in chromosome arm Xp, 9p, 15p, or 15q. As another example, the subject has an increased risk of breast cancer if the homologous telomere length difference (HTLD) is greater in chromosome arm 5q, Xp, 8q, 9p, 12p, 15p, or 15q than a reference homologous telomere length difference (HTLD). The reference HTLD can be, for example, the average, median, or quartile value of the HTLDs in cells from normal subjects of similar type to the cell. Any or a combination of parameters of chromosome telomere health can be used for such measurements.

The level of variability in telomere health within a cell (such as between the telomeres of a cell) can also be used to assess cancer risk. For example, pre-menopausal women have an increased risk of breast cancer if the within-cell telomere length variation (WCTLV) is less than a reference WCTLV, such as the WCTLV in normal subjects. As another example, pre-menopausal female have greater risk of breast cancer the lower the WCTLV. Any or a combination of parameters of chromosome telomere health can be used for such assessments. As another example, the subject has an increased risk of breast cancer if the within-cell telomere length variation (WCTLV) is greater than a reference within-cell telomere length variation (WCTLV). The reference WCTLV can be, for example, the average, median, or quartile value of the WCTLV in cells from normal subjects of similar type to the cell.

For these methods, it is useful to use the relative telomere health as the chromosome telomere health. Reference chromosome telomere healths preferably can be those of normal controls. In general, such normal controls can be similar chromosome telomere health parameter measurements made in unaffected subjects and cells. For example, the reference chromosome telomere health can be the average chromosome telomere health in cells from normal subjects of similar type to the cell, the average chromosome telomere health of the chromosome in cells from normal subjects of similar type to the cell, or the average arm-specific telomere health in cells from normal subjects of similar type to the cell. Any or a combination of parameters of chromosome telomere health can be used for such measurements. As another example, reference chromosome telomere health can be the median of the chromosome telomere health in cells from normal subjects of similar type to the cell being assessed, reference chromosome telomere health can be the median of the chromosome telomere health of the chromosome being assessed in cells from normal subjects of similar type to the cell being assessed, and reference chromosome telomere health can be the median of the arm-specific telomere lengths in cells from normal subjects of similar type to the cell being assessed. As another example, reference chromosome telomere health can be the quartile value of the chromosome telomere health in cells from normal subjects of similar type to the cell being assessed, reference chromosome telomere health can be the quartile value of the chromosome telomere health of the chromosome being assessed in cells from normal subjects of similar type to the cell being assessed, and reference chromosome telomere health can be the quartile value of the arm-specific telomere health in cells from normal subjects of similar type to the cell being assessed. In some forms, the parameter of the chromosome telomere health can be the relative telomere length. In some forms, the parameter of the chromosome telomere health can be the absolute telomere length. In some forms, the parameter of the chromosome telomere health can be the HTLD. In some forms, the parameter of the chromosome telomere health can be the WCTLV.

Also disclosed are methods of assaying a subject comprising measuring the length of at least one chromosome telomere of a chromosome in at least one cell of a sample from a subject, thereby producing a chromosome telomere length for at least one of the chromosome telomeres, and comparing the chromosome telomere length with a reference chromosome telomere length. By measuring individual chromosome telomeres and comparing to reference chromosome telomere lengths, cancer risk in subjects can be assessed.

For example, pre-menopausal women have an increased risk of breast cancer if the chromosome telomere length of chromosome telomere 1p-S, Xp-S, 9p-S, or 15p-S is shorter than the reference chromosome telomere length. As another example, post-menopausal women have an increased risk of breast cancer if the chromosome telomere length of chromosome telomere 15p-S is shorter than the reference chromosome telomere length. As another example, pre-menopausal women have greater risk of breast cancer the shorter the chromosome telomere length of chromosome telomere Xp-S or 15p-S is shorter than the reference chromosome telomere length.

Case-control comparison of mean RTLs identified four telomeres (1p-S, Xp-S, 9p-S and 15p-S) showed significant case-control difference at p<0.01 and one telomere (Xp-S) showed significant case-control difference at p<0.001 level in pre-menopausal women. In post-menopausal women, one telomere (15p-S) showed significant case-control difference at p<0.01 and none of the 46 telomeres showed significant case-control difference at p<0.001 level (Table 2).

Using the 50^{th} percentile value in controls as a cut point, multivariate logistic regression analysis confirmed that short telomere lengths on Xp-S and 15p-S were significantly associated with an increased breast cancer risk in premenopausal women, adjusted odds ratio (OR)=2.5 (95% CI=1.31 to 4.78) and 2.6 (95% CI=1.32 to 4.97) respectively (Table 3). ORs were adjusted for age, race, education, household income, physical activity in teens, smoking status, alcohol use and family history of cancer. When the study subjects were categorized into four groups (by quartiles) according to the telomere length, a highly significant inverse dose-response relationship was observed for Xp-S (P_{trend}=0.001) and 15p-S (P_{trend}=0.004), with the lowest-vs-highest quartile OR of 5.5 (95% CI=2.0 to 15.1) and 3.6 (95% CI=1.4 to 9.8) respectively (Table 3). In post-menopausal women, multivariate logistic regression analysis revealed that short telomere length on 15p-S was borderline significantly associated with an decreased breast cancer risk, adjusted OR=0.54 (95% CI=0.31 to 0.94). A significant dose-response relationship was also observed for 15p-S (P_{trend}=0.004, Table 3).

Shorter chromosome telomere lengths can be any length or degree shorter than the reference chromosome telomere length. For example, the chromosome telomere length can be a fraction of the reference chromosome telomere length. For example, the chromosome telomere length can be less than or equal to 0.5 of the reference chromosome telomere length.

Differences in the length of homologous telomeres (telomeres in homologous chromosome arms) can also be used to assess a subject's risk of cancer. For example, pre-menopausal women have an increased risk of breast cancer if the homologous telomere length difference (HTLD) is greater in chromosome arm 5q, Xp, 8q, 9p, 12p, 15p, or 15q than a reference HTLD, such as the HTLD for the chromosome arm in normal subjects. As another example, pre-menopausal women have a greater risk of breast cancer the greater the HTLD in chromosome arm Xp, 9p, 15p, or 15q. As another example, the subject has an increased risk of breast cancer if the homologous telomere length difference (HTLD) is greater in chromosome arm 5q, Xp, 8q, 9p, 12p, 15p, or 15q than a reference homologous telomere length difference (HTLD). The reference HTLD can be, for example, the average, median, or quartile value of the HTLDs in cells from normal subjects of similar type to the cell.

Case-control comparison of mean HTLD identified seven chromosome arms (5q, Xp, 8q, 9p, 12p, 15p and 15q) showed significant case-control difference at p-value<0.01 level and three chromosome arms (5q, 9p and 15p) showed significant case-control difference at p-value<0.001 level (significant after Bonferroni correction for multiple comparisons 0.05/46=0.0011) in pre-menopausal women (Table 4). None of the 46 chromosome arms showed significant case-control difference in post-menopausal women.

Using the 50^{th} percentile value in controls as a cut point, multivariate logistic regression analysis confirmed that greater difference in length between homologous telomeres on chromosome arms 9p, 15p and 15q were significantly associated with an increased breast cancer risk in premenopausal women, adjusted odds ratio (OR)=4.6 (95% CI=2.3 to 9.2), 3.1 (1.6 to 6.0) and 2.8 (1.4 to 5.4) respectively (Table 5). When the study subjects were categorized into four groups (by quartiles) according to the telomere length, a significant dose-response relationship was observed for chromosome Xp (P_{trend}=0.005), 9p (P_{trend}<0.001), 15p (P_{trend}<0.001) and 15q (P_{trend}=0.005), respectively (Table 5). None of the chromosome arms showed significant association with breast cancer risk in post-menopausal women (Table 10).

The level of variability in telomere length within a cell (such as between the telomeres of a cell) can also be used to assess cancer risk. For example, pre-menopausal women have an increased risk of breast cancer if the within-cell telomere length variation (WCTLV) is greater than a reference WCTLV, such as the WCTLV in normal subjects. As another example, pre-menopausal female have greater risk of breast cancer the greater the WCTLV. As another example, the subject has an increased risk of breast cancer if the within-cell telomere length variation (WCTLV) is greater than a reference within-cell telomere length variation (WCTLV). The reference WCTLV can be, for example, the average, median, or quartile value of the WCTLV in cells from normal subjects of similar type to the cell.

Telomere length variations among somatic cells (lymphocytes) were examined for their association with breast cancer risk. Fifteen to seventeen cells were assayed for each subject and standard deviations (SD) were computed for the RTL of each chromosome arm. The coefficient of variation (CV) was used, which is the adjusted SD (CV=SD/mean), as the measurement of telomere length variation because the value of SD is related to the mean RTL and mean RTL is associated with breast cancer risk. The average CV of 46 chromosome arms (homologous telomeres were combined) was found to be significantly higher in cases (mean CV=43.7%) than in controls (mean CV=41.9%, p=1.50×10⁻⁷) in pre-menopausal women (Table 6). The same level significant case-control differences in mean CV were also observed for homologous short version of the 46 telomeres (p=6.48×10⁻⁷) and homologous long version of the 46 telomeres (p=6.77×10⁻⁸) in pre-menopausal women. We did not observe any significant case-control difference in average CV of 46 chromosome arms in post-menopausal women (Table 6).

Case-control comparison of mean CV of each chromosome arm identified seven chromosome arms (lp-L, 5q-S, 12p-L, 15p-S, 18p-S, 18p-L and 19q-L) showed significant case-control difference at p<0.01 level and none of the mean CV of the 92 chromosome arms showed significant case-control difference at p<0.0005 level (Bonferroni correction 0.05/92=0.0005) in pre-menopausal women (Table 7). In post-menopausal women, two chromosome arms (21p-S and 21p-L) showed significant case-control difference at p<0.01 level and none of the 92 chromosome arms showed significant case-control difference at p<0.0005 level (Table 7). Using the 50^{th} percentile value in controls as a cut point, multivariate logistic regression analysis revealed suggestive associations between the greater telomere length variations on chromosome arms 1p-L, 18p-S and 19q-L and an increased breast cancer risk in premenopausal women, adjusted OR=2.6 (95% CI=1.3 to 5.0), 2.4 (1.3 to 4.6), and 2.5 (1.3 to 4.9) respectively (Table 8). When the study subjects were categorized into four groups (by quartiles) according to the telomere length, a significant dose-response relationship was observed for chromosome 18p-S (P_{trend}=0.003) (Table 8). In post-menopausal women, greater telomere length variations on chromosome arms 15p-S and 21p-L were associated with a decreased breast cancer risk, adjusted OR=0.47 (95% CI=0.27 to 0.82) and 0.44 (0.25 to 0.77) respectively (Table 8). When the study subjects were categorized into four groups (by quartiles) according to the telomere length, a significant inverse dose-response relationship was observed for chromosome 15p-S (P_{trend}=0.006), and 21p-L (P_{trend} p=0.005) (Table 8).

Also disclosed are methods of assaying a subject comprising, collecting a sample from the subject, measuring the length of the telomeres of the subject, calculating telomere ratios, telomere length variation, homologous telomere length difference, frequency of extremely long or short telomeres, wherein the 9p telomere ratio compares the length of the 9p telomere to the total length of the telomeres of all chromosomes of the subject, and identifying a subject having a 9p telomere ratio less than or equal to 0.00680 or alone or in any combination.

Also disclosed are methods, wherein the sample comprises a blood sample, wherein the 9p telomere length measured is the short arm, further comprising the step of identifying the subject as a subject having an increased risk of breast cancer, wherein the 9p telomere ratio is less than or equal to 0.00583, wherein the 9p telomere ratio is less than or equal to 0.00494, wherein collecting the sample comprises culturing lymphocytes within 48 hours after blood collection, wherein collecting the sample comprises harvesting the chromosomes with a cytogenic protocol, wherein measuring the length of the telomeres comprises using a fluorescent in situ hybridization assay, further comprising obtaining a patient history of the subject, further comprising adjusting the estimated cancer risk (odds ratio) based on one or more characteristics identified in the patient history, and/or wherein the odds ratio is analyzed, alone or in combination with other markers/host factors to predict cancer risk.

Disclosed are methods of assaying the risk of cancer, in a subject, based on telomere length comprising: taking a blood sample from the subject; harvesting the chromosomes; performing telomere analysis; quantitating telomere length; and determining the risk a subject of getting cancer if the subject's 9p telomere length is less than 0.680% or 0.583% or 0.494% of the length of all the subject's chromosomes telomere length alone or in combination with other markers/host factors.

Also disclosed are methods wherein cancer is breast cancer, wherein the chromosome 9p telomere is the shorter of the two 9p telomeres, 9p short, wherein telomere analysis comprises fluorescent in situ hybridization (FISH) analysis, wherein quantitating telomere length comprises quantifying the fluorescence using TeloMeter or Isis software.

Also disclosed are methods of treating a subject, such as a patient comprising, analyzing the results of the method of any of the methods disclosed herein, and performing a treatment treat or to prevent cancer on the subject based on the results of the method alone or in combination with other markers/host factors.

A method of assaying a subject comprising, collecting a sample from the subject and setting up a blood culture, performing a telomere fluorescent in situ hybridization, measuring the intensity of the telomere fluorescent signals of the hybridization from the sample of the subject, creating a telomere ratio, wherein the relative telomere length of the short arms of chromosome 9 (9p) is the ratio of the telomere signal intensity of the 9p to the telomere signal intensity of all the telomeres in a cell from the sample, and identifying a subject having a 9p telomere ratio less than or equal to a reference 9p telomere ratio or alone or in any combination with any other aspects disclosed herein.

Also disclosed are methods, wherein the sample comprises a blood sample, wherein the 9P telomere length measured is the short arm of chromosome #9, further comprising the step of identifying the subject as a subject having an increased risk of breast cancer, wherein the reference 9P telomere ratio is less than or equal to 0.680%, wherein the reference 9P telomere ratio is less than or equal to 0.583%, wherein the reference 9P telomere ratio is less than or equal to 0.494%, wherein setting up the lymphocyte culture within 48 hours of blood sample collection, wherein collecting the sample comprises culturing blood lymphocytes (see for example, Zheng et al, 2003, Carcinogenesis 24:269-74), wherein collecting the sample comprises harvesting the chromosomes with a cytogenic protocol (see for example, Zheng et al, 2003, Carcinogenesis 24:269-74), wherein measuring the length of the telomeres comprises using a fluorescence in situ hybridization (FISH) assay, wherein measuring the length of the telomeres comprises analyzing FISH images of metaphase chromosomes using the TeloMeter, further comprising obtaining a demographic, lifestyle and medical history of the subject, wherein the statistical analysis indicates at least a 3.0, 3.9, or 6.6, fold increase relative to all women, wherein the statistical analysis indicates at least a 2.1, 2.9, or 6.2, fold increase relative to all premenopausal women, and/or wherein the statistical analysis indicates at least a 4.3, 5.1, or 7.5, fold increase relative to all postmenopausal women, further comprising a statistical analysis (logistic regression modeling) to predict the likelihood of having breast cancer using the relative telomere length of chromosome 9p alone as the predictor or in combination with other markers/host factors to predict cancer risk alone or in any combination with any other aspects disclosed herein.

Disclosed are methods wherein relative to all women, the likelihood of having breast cancer given having shorter telomere length (0.583%-0.680%) on 9p increases 3.0-fold when compared to women having long telomere (>0.680%) on 9p, wherein relative to all women, wherein the likelihood of having breast cancer given having shorter telomere length (0.494%-0.583%) on 9p increases 3.9-fold when compared to women having long telomere (>0.680%) on 9p, wherein relative to all women, wherein the likelihood of having breast cancer given having shorter telomere length (<0.494%) on 9p increases 6.6-fold when compared to women having long telomere (>0.680%) on 9p, wherein relative to pre-menopausal women, wherein the likelihood of having breast cancer given having shorter telomere length (0.583%4680%) on 9p increases 2.1-fold when compared to women having long telomere (>0.680%) on 9p, wherein relative to pre-menopausal women, wherein the likelihood of having breast cancer given having shorter telomere length (0.494%-0.583%) on 9p increases 2.9-fold when compared to women having long telomere (>0.680%) on 9p, wherein relative to pre-menopausal women, wherein the likelihood of having breast cancer given having shorter telomere length (<0.494%) on 9p increases 6.2-fold when compared to women having long telomere (>0.680%) on 9p, wherein relative to post-menopausal women, wherein the likelihood of having breast cancer given having shorter telomere length (0.583%-0.680%) on 9p increases 4.3-fold when compared to women having long telomere (>0.680%) on 9p, wherein relative to post-menopausal women, wherein the likelihood of having breast cancer given having shorter telomere length (0.494%-0.583%) on 9p increases 5.1-fold when compared to women having long telomere (>0.680%) on 9p, and/or wherein relative to post-menopausal women, wherein the likelihood of having breast cancer given having shorter telomere length (<0.494%) on 9p increases 7.5-fold when compared to women having long telomere (>0.680%) on 9p alone or in any combination with any other aspects disclosed herein.

In certain embodiments, the methods address the relationship between length and cancer risk, such as breast cancer risk, that for every 10% decrease in relative telomere length of 9p, there is a 37% (95% CI=20%-57%, p<0.0001) increase in cancer risk, such as breast cancer risk. Thus, a step that can be added to any of the disclosed methods is a step of determining % decrease, or any equivalent operation, such as relative amounts, of the relative telomere length, or telomere ratio, and additionally, a step of converting this decrease to a risk assessment for breast cancer based on the disclosed relationship. Generally the idea of an increased risk of breast cancer based on a decrease in telomere length is disclosed.

Disclosed are methods of assaying the risk of cancer, in a subject, based on telomere length comprising: taking a blood sample from the subject; Setting up a lymphocyte culture using fresh whole blood; harvesting the chromosome preparation using standard cytogenetic method; performing quantitative telomere fluorescent in situ hybridization (FISH); analyzing FISH images of chromosome spreads to quantify telomere length (telomere signal intensity); defining the relative telomere length of chromosome 9p as the intensity of 9p telomere signal divided by the intensity of total telomere signals of the cell; determining the likelihood of getting cancer for a individual using statistic prediction model considering if the individual's 9p telomere length is less than a specific cut points (0.494%, 0.583% or 0.680%) or alone or in combination with other markers/host factors, alone or in any combination with any other aspects disclosed herein.

Also disclosed are methods, wherein cancer is breast cancer, wherein the chromosome 9p telomere is the shorter of the two 9p telomeres (9p-short), wherein the two of the chromosome 9p telomeres can be used jointly to predict cancer risk, wherein chromosome analysis comprises fluorescent in situ hybridization (FISH) analysis, wherein quantitating telomere length comprises quantifying the intensity of the fluorescence signal using TeloMeter or Isis image software or alone or in any combination.

Variation in telomere length and the frequency of extremely short telomeres, which indicate telomere health, are strongly associated with lung cancer risk. In some forms, greater telomere length variation, greater frequency of extremely short telomeres, or both, indicate a risk of lung cancer. In some forms, variation in telomere length and the frequency of extremely short telomeres, or both, in subjects 60 years of age or younger indicate a risk of lung cancer risk.

In some forms, telomere length variation over median telomere length variation, frequency of extremely short telomeres over median frequency of extremely short telomeres, or both, indicate a risk of lung cancer. In some forms, telomere length variation over median telomere length variation, frequency of extremely short telomeres over median frequency of extremely short telomeres, or both, in subjects 60 years of age or younger indicate a risk of lung cancer.

In some forms, telomere length variation in the highest quartile of telomere length variation, frequency of extremely short telomeres in the highest quartile of frequency of extremely short telomeres, or both, indicate a risk of lung cancer. In some forms, telomere length variation in the highest quartile of telomere length variation, frequency of extremely short telomeres in the highest quartile of frequency of extremely short telomeres, or both, in subjects 60 years of age or younger indicate a risk of lung cancer.

The data in the Examples show significant correlations of telomere length variation and percent of short telomeres to the risk of lung cancer in subject 60 years of age and younger. As before, short telomeres were defined as telomeres that were shorter than 10% of the average telomere length. The data show that, for subjects 60 years old and younger, a telomere length variation over the median telomere length variation (TLV of 65.0) indicates a risk of lung cancer (odds ratio of 6.65 and p=0.0031). The data also show that, for subjects 60 years old and younger, a telomere length variation in the highest quartile (TLV of 69.2-78.7; TLV over 68.7) indicates a risk of lung cancer (odds ratio of 16.06 and p=0.0039). The data also show that, for subjects 60 years old and younger, a percent of short telomeres over the median percent of short telomeres (3.44% of short telomeres; 0.0344 frequency of extremely short telomeres) indicates a risk of lung cancer (odds ratio of 5.11 and p=0.0044). The data also show that, for subjects 60 years old and younger, a percent of short telomeres in the highest quartile (4.89-7.64% of short telomeres; 0.0489-0.0764 frequency of extremely short telomeres) indicates a risk of lung cancer (odds ratio of 25.17 and p=0.0051).

Also disclosed are methods of treating a patient comprising, analyzing the results of the method of any of the methods disclosed herein alone or in combination with other markers/host factors to predict cancer risk, and based on the risk performing a preventive treatment/intervention for cancer on the subject.

For these methods, it is useful to use the relative telomere length as the chromosome telomere length. It is also useful to use the absolute telomere length as the chromosome telomere length. Reference chromosome telomere lengths preferably can be normal controls. In general, such normal controls can be similar chromosome telomere length measurements made in unaffected subjects and cells. For example, the reference chromosome telomere length can be the average chromosome telomere length in cells from normal subjects of similar type to the cell, the average chromosome telomere length of the chromosome in cells from normal subjects of similar type to the cell, or the average arm-specific telomere length in cells from normal subjects of similar type to the cell. As another example, reference chromosome telomere length can be the median of the chromosome telomere lengths in cells from normal subjects of similar type to the cell being assessed, reference chromosome telomere length can be the median of the chromosome telomere lengths of the chromosome being assessed in cells from normal subjects of similar type to the cell being assessed, and reference chromosome telomere length can be the median of the arm-specific telomere lengths in cells from normal subjects of similar type to the cell being assessed. As another example, reference chromosome telomere length can be the quartile value of the chromosome telomere lengths in cells from normal subjects of similar type to the cell being assessed, reference chromosome telomere length can be the quartile value of the chromosome telomere lengths of the chromosome being assessed in cells from normal subjects of similar type to the cell being assessed, and reference chromosome telomere length can be the quartile value of the arm-specific telomere lengths in cells from normal subjects of similar type to the cell being assessed.

Any suitable techniques can be used to measure parameters of chromosome telomere health. Any suitable techniques can be used to measure the length of chromosome telomeres. For example, chromosome telomere length can be measured by obtaining the sample from the subject, where the sample is a blood sample, for example; harvesting the chromosome from at least one cell in the blood sample; performing telomere analysis; and quantitating telomere length. Telomere analysis and quantitating telomere length can also be accomplished using any suitable techniques. For example, telomere analysis and quantitating telomere length can be accomplished by telomere quantitative fluorescent in situ hybridization (QT-FISH). Useful for quantitating telomere length are techniques that total the fluorescent signal from telomere probes from the chromosome telomere of interest.

### VIII. Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6.times.SSC or 6.times.SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### IX. Actions Based on Identifications

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, subjects are identified as having a higher or lower risk of local recurrence of cancer and appropriate treatments are identified based on such risk identifications. Such identifications are useful for many reasons. For example, and in particular, such identifications allow specific actions to be taken based on, and relevant to, the particular identification made. For example, prognosis of a particular disease or condition in particular subjects (and the lack of diagnosis of that disease or condition in other subjects) has the very useful effect of identifying subjects that would benefit from treatment, actions, behaviors, etc. based on the prognosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising making one or more further identifications based on one or more other identifications. For example, particular treatments, monitorings, follow-ups, advice, etc. can be identified based on the other identification. For example, identification of a subject as having a disease or condition with a high level of a particular component or characteristic can be further identified as a subject that could or should be treated with a therapy based on or directed to the high level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods comprising treating, monitoring, following-up with, advising, etc., a subject identified in any of the disclosed methods. Also disclosed are methods comprising treating, monitoring, following-up with, advising, etc., a subject for which a record of an identification from any of the disclosed methods has been made. For example, particular treatments, monitorings, follow-ups, advice, etc. can be used based on an identification and/or based on a record of an identification. For example, a subject identified as having a disease or condition with a high level of a particular component or characteristic (and/or a subject for which a record has been made of such an identification) can be treated with a therapy based on or directed to the high level component or characteristic. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

### EXAMPLES

### Example 1

### Correlation Analysis of Breast Cancer Risk with Relative Telomere Length, Homologous Telomere Length Difference, and Within-cell Telomere Length Variation

This example describes the first genome-wide telomere association study to examine the associations between lengths of 92 telomeres in blood lymphocytes and breast cancer risk. The correlations discovered indicate roles of chromosomal telomeres in breast cancer susceptibility and provide the foundation of the disclosed methods.

### I. Methods

A. Study Population

The study was approved by the MedStar Research Institute-Georgetown University Oncology Institutional Review Board. The details of study population were described previously (Zheng, 2009). Breast cancer cases (N=204) were recruited at the Georgetown University Medical Center clinics (Lombardi Comprehensive Cancer Center's Division of Medical Oncology, Department of Surgery and the Betty Lou Ourisman Breast Cancer Clinic). The inclusion criteria for cases included a diagnosis of breast cancer within the prior 6 months, women, have not been treated yet with chemotherapy and radiotherapy, ability to provide informed consent in English. Exclusion criteria were women with a prior history of cancer, had chemotherapy and radiation treatment, or had active infection or immunological disorder that needed to be treated with antibiotics or immunosuppressive medication within the prior one month. The overall participation rate among eligible patients is 70%.

Controls (N=236) were randomly selected from healthy women who visited the mammography screening clinic at Georgetown University Medical Center, frequency matched to cases by age (2-year interval), race, and state of residency (D.C., Maryland or Virginia). Other inclusion and exclusion criteria for controls were the same as for cases. Additionally, women who had breast biopsy or were pregnant or breast feeding were not eligible. The overall participation rate among the eligible women was 60% for controls. After providing informed consent, subjects received a structured, in-person interview assessing prior medical history, tobacco smoke exposures, alcohol use, current medications, family medical history, reproductive history, and socioeconomic characteristics. Venous blood was obtained by trained interviewers using heparinized tubes.

B. Chromosome Preparation from Blood Cultures

Short-term lymphocyte cultures were established from fresh blood within 48 hours after the samples were obtained, as previously described (Zheng, 2005). One ml of fresh whole blood was added to 9 ml of RPMI-1640 medium supplemented with 15% fetal bovine serum, 1.5% of phytohemagglutinin, 2 mM L-glutamine, and 100 U/ml each of penicillin and streptomycin. Cells were cultured for 4 days at 37° C. and colcemid (0.2 μg/ml) was added to the culture 1 hour before the harvest. The cells were treated in hypotonic solution (0.06 M KCl) at room temperature (RT) for 25 mins, fixed in crayon fixative (methanol:acetic acid=3:1), and kept in crayon fixative at −20° C.

C. Telomere Length Measurement and Quality Control

Chromosome arm-specific telomere lengths were measured by telomere quantitative fluorescent in situ hybridization (TQ-FISH) in combination with karyotyping by DAPI-banding (equivalent to G-banding). The chromosome preparations were dropped onto clean microscopic slides and fixed in crayon fixative for one hour, dehydrated through an ethanol series (70%, 80%, 90% and 100%), and air dried. Fifteen microliters of hybridization mixture consisting of 0.3 μg/ml Cy3-labeled telomere-specific peptide nucleic acid (PNA) probe (Panagene Inc., Daej eon, Korea), 1 ml of cocktails of FITC-labeled centromeric PNA probes specific for chromosomes 2, 4, 8, 9, 13, 15, 18, 20 and 21 (Biomarkers, Rockville, Md.), 20 mg/ml of Cy3-labeled centromeric PNA probes specific for chromosome X, 50% formamide, 10 mM Tris-HCl, pH 7.5, 5% blocking reagent, and 1× Denhart's solution was applied to each slide. Slides were then placed in a Hybex microarray hybridization oven (SciGene, Sunnyvale, Calif.) where the DNA was denatured by incubating at 75° C. for five minutes, followed by hybridizing at 30° C. for three hours. After hybridization, the slides were sequentially washed; once in 1×SSC, once in 0.5×SSC, and once in 0.1×SSC; each wash was 10 min at 42° C. The slides were then mounted in anti-fade mounting medium containing 300 ng/ml 4′-6-diamidino-2-phenylindole (DAPI).

After TQ-FISH, cells were analyzed using an epifluorescence microscope equipped with a charge-coupled device (CCD) camera (Leica Microsystems, Bannockburn, Ill.). Images were captured with exposure times of 0.15, 0.25 and 0.05 second for Cy3, FITC and DAPI signals, respectively. Digitized images were analyzed using a specialized Isis FISH imaging software with a telomere module (MetaSystems Inc. Boston, Mass.). This software permits measurement of 92 telomere signals simultaneously after karyotyping. Chromosome identification was achieved by: (1) DAPI banding (equivalent to G-banding); (2) chromosome specific centromere probes. Arm-specific telomere length measurements were made by telomere fluorescent in situ hybridization. Telomere sequences were labeled by a Cy3 (red) telomere specific PNA probe. Chromosomes 2, 4, 8, 9, 13, 15, 18, 20 and 21 were identified by combination of DAPI banding and chromosome specific centromeric probe (green signals). Chromosome X was identified by a Cy3 (red) labeled centromere probe.

Telomere fluorescent intensity units (FIU) were recorded as an indirect measurement of telomere length. Between the homologous telomeres, one telomere was often shorter than the other and there are significant differences in lengths between homologous telomeres. This observation is consistent with previous reports indicating that arm-specific telomere lengths were highly variable between chromosome arms and between homologous arms (Gilson, 2007; Graakjaer, 2006). Thus, each pair of homologous telomeres was recorded separately as homologous short (S) and homologous long (L). To normalize the FISH hybridization variations between samples, relative telomere length (RTL) was used for the subsequent statistical analysis. For each study subject, 15-17 metaphase cells were analyzed to estimate the mean relative telomere length for the 92 telomeres.

The definition of telomere-related parameters were as following: (1) relative telomere length (RTL) was defined as the ratio of the arm-specific telomere FIU to the total telomere FIU of 92 telomeres from the same cell; (2) homologous telomere length difference (HTLD) was defined as the percent of (homologous long TL−homologous short TL) divided by (homologous long TL+homologous short TL); (3) coefficient of variation (CV) of TL among 92 telomeres in a typical human cell was used as the measurement of telomere length variation.

Several quality control steps were used in this assay. Laboratory personnel who were responsible for the blood culture and telomere assay were blinded to the case-control status of the subjects. All new lots of reagents were tested to ensure optimal hybridization. A control slide containing cells with known total telomere length was included in each batch of TQ-FISH to monitor the quality of the hybridization efficiency. In this study, the coefficient of variation (CV) of overall telomere length from 20 repeats of the control slide was 12.4% and the correlations of telomere lengths between repeated samples were 0.89.

D. Statistical Analysis

Student t-test was used to compare the means of the variables (telomere length, homologous telomere length variation or telomere length variation in somatic cells) that were normally distributed and Wilcoxon rank sum test was used for non-normally distributed variables. Chi-square tests were used to compare the distribution of categorical variables between cases and controls. Pearson correlation was used to examine the correlations between telomere lengths, and between telomere lengths and age.

The associations between telomere-related parameters and the risk of breast cancer were examined using unconditional logistic regression. Relative telomere lengths, homologous telomere length variation and overall telomere length variation were dichotomized as short/long or high/low using the 50^{th} percentile values in the controls as a cut point. Telomere lengths were also categorized according to the quartiles in control subjects. Odds ratios were adjusted for age, race, smoking status, alcohol use, education, family history of cancer in first degree biological relatives, menopausal status, physical activity in the teens. P-values were two-sided and considered statistically significant if P<0.01. All analyses were performed using SAS software, version 9 (SAS Institute Inc., Cary, N.C.).

### II. Results

A. Characteristics of Study Population

Table 1 lists the characteristics of the study subjects. The mean age is 53.0 for cases and 53.2 for controls. There are no significant case-control differences in the distributions of race, menopausal status, tobacco smoking status, alcohol use, education levels, family history of cancer, levels of household income and HRT use. The mean body mass index (BMI) was similar between cases and controls. Controls were significantly more likely to be physically active in both the teens and in the past year compared with cases. The mean age at first live birth is older in controls than in cases (Table 1).

TABLE 1
Distribution of Characteristics and Known Breast Cancer Risk
Factors

| | Cases | Controls | |
|---|---|---|---|
| | N = 205 | N = 236 | p-value |
| Demographic factors† | | | |
| Age (y) | 53.0 ± 11.0 | 53.2 ± 10.0 | 0.853 |
| Race, N (%) | | | |
| White | 74.1 | 71.5 | |
| Black | 20.3 | 25.0 | |
| Other | 5.6 | 3.5 | 0.343 |
| Education ≧ college (%) | 40.9 | 43.4 | 0.610 |
| Household Income ≧ 100K (%) | 55.0 | 56.1 | 0.850 |
| Reproductive risk factors† | | | |
| Age at menarche (y) | 12.6 ± 1.5 | 12.5 ± 1.8 | 0.583 |
| Postmenopausal (%) | 55.3 | 57.9 | 0.586 |
| Number of live births | 1.53 ± 1.31 | 1.51 ± 1.29 | 0.852 |
| Age at first live birth (y) | 27.1 ± 6.3 | 28.8 ± 6.6 | 0.042 |
| Used HRT‡ (%) | 32.8 | 39.8 | 0.138 |
| Other risk factors† | | | |
| Had FHC (%) | 57.8 | 52.3 | 0.268 |
| Body mass index | 27.3 ± 6.5 | 27.3 ± 7.1 | 0.970 |
| Ever smoked cigarettes (%) | 37.6 | 46.2 | 0.072 |
| Ever drank Alcohol (%) | 88.7 | 92.0 | 0.246 |
| Exercised regularly at teens (%) | 66.5 | 80.9 | <0.001 |

†Unless otherwise specified, mean ± SD are presented.
‡HRT = hormonal replacement therapy.

Exercised regularly was defined as any weekly physical activity that would make the subject sweat or increase their heart rate and last >20 minutes.
Family history of cancer (FHC) was defined as any cancer cases among 1^{st} degree blood relatives.

B. Telomere Length Correlation Between Chromosome Arms

Whether telomere length was correlated between chromosomal arms in control subjects was evaluated and it was determined that lengths between homologous telomeres were significantly correlated, Pearson correlation coefficient (r) ranged from 0.39 to 0.66 for 46 pairs of homologous telomeres (mean=0.52), all p-values <0.0001 (significant after Bonferroni correction for multiple comparisons 0.05/46=0.0011). In contrast, telomere lengths between non-homologous telomeres were not correlated, Pearson correlation coefficient ranged from −0.19 to 0.21, none of the p-value reached statistical significance after adjustment for multiple comparisons. Correlation between arm-specific telomere length and patient's age in control subjects was also examined Significant correlations were observed for chromosome 2p-S (r=−0.22, p=0.0007) and 15p-S (r=−0.24, p=0.0003).

C. Arm-Specific Telomere Length and Breast Cancer Risk

Initial case-control comparison of mean RTLs identified four telomeres (1p-S, Xp-S, 9p-S and 15p-S) showed significant case-control difference at p<0.01 and one telomere (Xp-S) showed significant case-control difference at p<0.001 level (significant after Bonferroni correction for multiple comparisons 0.05/46=0.0011) in pre-menopausal women. In post-menopausal women, one telomere (15p-S) showed significant case-control difference at p<0.01 and none of the 46 telomeres showed significant case-control difference at p<0.001 level (Table 2). It is important to note that none of the homologous long version of the telomeres showed significant case-control differences (Table 9).

Because telomere lengths between homologous telomeres are significantly correlated, the analyses were focused on homologous short version of the 46 telomeres. Using the 50^{th} percentile value in controls as a cut point, multivariate logistic regression analysis confirmed that short telomere lengths on Xp-S and 15p-S were significantly associated with an increased breast cancer risk in premenopausal women, adjusted odds ratio (OR)=2.5 (95% CI=1.31 to 4.78) and 2.6 (95% CI=1.32 to 4.97) respectively (Table 3). ORs were adjusted for age, race, education, household income, physical activity in teens, smoking status, alcohol use and family history of cancer. When the study subjects were categorized into four groups (by quartiles) according to the telomere length, a highly significant inverse dose-response relationship was observed for Xp-S (P_{trend}=0.001) and 15p-S (P_{trend}=0.004), with the lowest-vs-highest quartile OR of 5.5 (95% CI=2.0 to 15.1) and 3.6 (95% CI=1.4 to 9.8) respectively (Table 3). In post-menopausal women, multivariate logistic regression analysis revealed that short telomere length on 15p-S was borderline significantly associated with an decreased breast cancer risk, adjusted OR=0.54 (95% CI=0.31 to 0.94). A significant dose-response relationship was also observed for 15p-S (P_{trend}=0.004, Table 3).

D. Telomere Length Variation Between Homologous Telomeres and Breast Cancer Risk

To test the hypothesis that increased telomere length variations across the genome will increase genomic instability, hence increased risk of breast cancer, whether differences in length between homologous telomeres are associated with breast cancer risk were examined. Homologous telomere length difference (HTLD) was defined as the percent of (homologous long TL−homologous short TL) divided by (homologous long TL+homologous short TL). Initial case-control comparison of mean HTLD identified seven chromosome arms (5q, Xp, 8q, 9p, 12p, 15p and 15q) showed significant case-control difference at p-value <0.01 level and three chromosome arms (5q, 9p and 15p) showed significant case-control difference at p-value <0.001 level (significant after Bonferroni correction for multiple comparisons 0.05/46=0.0011) in pre-menopausal women (Table 4). None of the 46 chromosome arms showed significant case-control difference in post-menopausal women.

Using the 50^{th} percentile value in controls as a cut point, multivariate logistic regression analysis confirmed that greater difference in length between homologous telomeres on chromosome arms 9p, 15p and 15q were significantly associated with an increased breast cancer risk in premenopausal women, adjusted odds ratio (OR)=4.6 (95% CI=2.3 to 9.2), 3.1 (1.6 to 6.0) and 2.8 (1.4 to 5.4) respectively (Table 5). When the study subjects were categorized into four groups (by quartiles) according to the telomere length, a significant dose-response relationship was observed for chromosome Xp (P_{trend}=0.005), 9p (P_{trend}<0.000, 15p (P_{trend}<0.001) and 15q (P_{trend}=0.005), respectively (Table 5). None of the chromosome arms showed significant association with breast cancer risk in post-menopausal women (Table 10).

E. Chromosome Arm-Specific Telomere Length Variation in Lymphocytes and Breast Cancer Risk

Telomere length variations among somatic cells (lymphocytes) were examined for their association with breast cancer risk. Fifteen to seventeen cells were assayed for each subject and standard deviations (SD) were computed for the RTL of each chromosome arm. The coefficient variation (CV) was used, which is the adjusted SD (CV=SD/mean), as the measurement of telomere length variation because the value of SD is related to the mean RTL and mean RTL is associated with breast cancer risk. The average CV of 46 chromosome arms (homologous telomeres were combined) was found to be significantly higher in cases (mean CV=43.7%) than in controls (mean CV=41.9%, p=1.50×10⁻⁷) in pre-menopausal women (Table 6). The same level significant case-control differences in mean CV were also observed for homologous short version of the 46 telomeres (p=6.48×10⁻⁷) and homologous long version of the 46 telomeres (p=6.77×10⁻⁸) in pre-menopausal women. We did not observe any significant case-control difference in average CV of 46 chromosome arms in post-menopausal women (Table 6).

Case-control comparison of mean CV of each chromosome arm identified seven chromosome arms (lp-L, 5q-S, 12p-L, 15p-S, 18p-S, 18p-L and 19q-L) showed significant case-control difference at p<0.01 level and none of the mean CV of the 92 chromosome arms showed significant case-control difference at p<0.0005 level (Bonferroni correction 0.05/92=0.0005) in pre-menopausal women (Table 7). In post-menopausal women, two chromosome arms (21p-S and 21p-L) showed significant case-control difference at p<0.01 level and none of the 92 chromosome arms showed significant case-control difference at p<0.0005 level (Table 7). Using the 50^{th} percentile value in controls as a cut point, multivariate logistic regression analysis revealed suggestive associations between the greater telomere length variations on chromosome arms 1p-L, 18p-S and 19q-L and an increased breast cancer risk in premenopausal women, adjusted OR=2.6 (95% CI=1.3 to 5.0), 2.4 (1.3 to 4.6), and 2.5 (1.3 to 4.9) respectively (Table 8). When the study subjects were categorized into four groups (by quartiles) according to the telomere length, a significant dose-response relationship was observed for chromosome 18p-S (P_{trend}=0.003) (Table 8). In post-menopausal women, greater telomere length variations on chromosome arms 15p-S and 21p-L were associated with a decreased breast cancer risk, adjusted OR=0.47 (95% CI=0.27 to 0.82) and 0.44 (0.25 to 0.77) respectively (Table 8). When the study subjects were categorized into four groups (by quartiles) according to the telomere length, a significant inverse dose-response relationship was observed for chromosome 15p-S (P_{trend}=0.006), and 21p-L (P_{trend} p=0.005) (Table 8).

TABLE 2
Case-Control Comparison of Mean Relative Telomere Length (RTL) on homologous
short version of chromosome arms

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p-value† | N = 89 | N = 96 | p-value† | N = 110 | N = 132 | p-value† |
| 1p | 0.749 | 0.777 | 0.0432 | 0.744 | 0.805 | 0.0027 | 0.751 | 0.759 | 0.6687 |
| Xp | 0.783 | 0.802 | 0.2193 | 0.759 | 0.834 | 0.0008 | 0.780 | 0.778 | 0.2852 |
| 9p | 0.663 | 0.692 | 0.0239 | 0.665 | 0.719 | 0.0069 | 0.660 | 0.673 | 0.4449 |
| 15p | 0.700 | 0.697 | 0.7968 | 0.682 | 0.740 | 0.0073 | 0.713 | 0.664 | 0.0078 |

TABLE 3
Logistic Regression Examining the Association between Homologous
Short RTL and Breast Cancer Risk

| | | | Pre-menopausal | | Post-menopausal | |
|---|---|---|---|---|---|---|
| Chromosome | All subjects N = 440 | | women N = 185 | | women N = 242 | |
| arm | OR (95% CI) | p | OR (95% CI) | p | OR (95% CI) | p |
| 1p | | | | | | |
| By median | 1.35 (0.89-2.03) | 0.1549 | 1.73 (0.90-3.31) | 0.0994 | 1.08 (0.62-1.87) | 0.7869 |
| By quartiles | | | | | | |
| Q3 | 1.14 (0.63-2.05) | | 1.97 (0.80-4.84) | | 0.75 (0.33-1.71) | |
| Q2 | 1.15 (0.64-2.08) | | 1.96 (0.76-4.95) | | 0.72 (0.32-1.60) | |
| Q1 | 1.76 (0.99-3.13) | 0.0597* | 3.07 (1.20-7.86) | 0.0264* | 1.15 (0.54-2.45) | 0.6494* |
| Xp | | | | | | |
| By median | 1.12 (0.75-1.67) | 0.5920 | 2.50 (1.31-4.78) | 0.0055 | 0.59 (0.34-1.02) | 0.0603 |
| By quartiles | | | | | | |
| Q3 | 1.61 (0.90-2.86) | | 3.38 (1.31-8.69) | | 0.90 (0.40-2.02) | |
| Q2 | 1.17 (0.64-2.12) | | 5.21 (1.84-14.78) | | 0.37 (0.16-0.85) | |
| Q1 | 1.64 (0.92-2.91) | 0.2154* | 5.45 (1.97-15.05) | 0.0010* | 0.69 (0.32-1.49) | 0.1528* |
| 9p | | | | | | |
| By median | 1.13 (0.75-1.69) | 0.5632 | 1.42 (0.75-2.69) | 0.2764 | 1.02 (0.59-1.78) | 0.9366 |
| By quartiles | | | | | | |
| Q3 | 2.18 (1.21-3.91) | | 3.14 (1.28-7.72) | | 1.55 (0.68-3.54) | |
| Q2 | 1.55 (0.84-2.84) | | 2.23 (0.89-5.61) | | 1.27 (0.54-2.98) | |
| Q1 | 1.88 (1.04-3.41) | 0.1400* | 2.54 (1.00-6.43) | 0.0860* | 1.37 (0.61-3.08) | 0.6470* |
| 15p | | | | | | |
| By median | 1.05 (0.70-1.57) | 0.8318 | 2.56 (1.32-4.97) | 0.0054 | 0.54 (0.31-0.94) | 0.0283 |
| By quartiles | | | | | | |
| Q3 | 0.75 (0.42-1.33) | | 1.64 (0.67-4.03) | | 0.38 (0.17-0.87) | |
| Q2 | 1.02 (0.58-1.77) | | 2.99 (1.23-7.26) | | 0.41 (0.18-0.91) | |
| Q1 | 0.82 (0.46-1.44) | 0.7203* | 3.63 (1.35-9.75) | 0.0035* | 0.30 (0.14-0.64) | 0.0039* |

*p-for-trend
Bold p-values are significant at <0.01 level.
ORs were adjusted for age, race, education, household income, physical activity in teens, smoking status, alcohol use, family history of cancer and history of pregnancy.

TABLE 4
Case-Control Comparison of Mean Homologous Telomere Length Differences (HTLD)

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p-value† | N = 89 | N = 96 | p-value† | N = 110 | N = 132 | p-value† |
| 5q | 38.45 | 36.21 | 0.0013 | 39.39 | 35.60 | 0.0006 | 37.93 | 36.61 | 0.1509 |
| Xp | 38.27 | 37.16 | 0.1280 | 39.37 | 36.40 | 0.0076 | 37.62 | 37.95 | 0.7445 |
| 8q | 40.68 | 38.48 | 0.0036 | 40.93 | 38.00 | 0.0077 | 40.80 | 38.90 | 0.0771 |
| 9p | 38.91 | 37.14 | 0.0169* | 39.10 | 35.90 | 0.0005* | 38.95 | 38.11 | 0.6494* |
| 12p | 38.80 | 36.86 | 0.0081 | 39.76 | 36.55 | 0.0058 | 38.18 | 37.30 | 0.3627 |
| 15p | 38.95 | 38.19 | 0.3038 | 40.14 | 35.87 | 0.0001 | 38.08 | 40.00 | 0.0533 |
| 15q | 38.13 | 36.55 | 0.0295 | 38.16 | 34.87 | 0.0034 | 38.20 | 37.99 | 0.8240 |

HTLD was defined as the percent of (homologous long T − homologous short T) divided by (homologous long T + homologous short T).
†all p-values were from t-test except for 9p.
*Wilcoxon rank sum test was used.
Bold p-values are significant after adjustment for multiple comparison (Bonferroni correction 0.05/46 = 0.0011).

TABLE 5
Logistic Regression Examining the Association between Allelic Telomere
Length Differences and Breast Cancer Risk

| | | | Pre-menopausal | | Post-menopausal | |
|---|---|---|---|---|---|---|
| Chromosome | All subjects N = 440 | | women N = 185 | | women N = 242 | |
| arm | OR (95% CI) | p | OR (95% CI) | p | OR (95% CI) | p |
| 5q | | | | | | |
| By median | 1.56 (1.03-2.36) | 0.0356 | 1.92 (1.00-3.71) | 0.0508 | 1.47 (0.84-2.58) | 0.1817 |
| By quartiles | | | | | | |
| Q2 | 1.10 (0.63-1.89) | | 1.21 (0.51-2.83) | | 0.97 (0.46-2.04) | |
| Q3 | 1.49 (0.84-2.64) | | 1.41 (0.60-3.34) | | 1.75 (0.79-3.91) | |
| Q4 | 1.87 (1.05-3.32) | 0.0218 | 3.43 (1.32-8.88) | 0.0169 | 1.31 (0.61-2.85) | 0.2760 |
| Xp | | | | | | |
| By median | 1.31 (0.87-1.97) | 0.1904 | 1.82 (0.95-3.49) | 0.0727 | 1.08 (0.63-1.88) | 0.7719 |
| By quartiles | | | | | | |
| Q2 | 1.24 (0.71-2.18) | | 2.94 (1.15-7.55) | | 0.64 (0.30-1.38) | |
| Q3 | 1.24 (0.71-2.17) | | 2.16 (0.87-5.37) | | 0.84 (0.39-1.81) | |
| Q4 | 1.81 (1.02-3.21) | 0.0561 | 3.98 (1.63-9.70) | 0.0048 | 0.89 (0.40-2.00) | 0.9413 |
| 8q | | | | | | |
| By median | 1.37 (0.91-2.06) | 0.1283 | 1.63 (0.87-3.05) | 0.1311 | 1.26 (0.73-2.19) | 0.4089 |
| By quartiles | | | | | | |
| Q2 | 2.53 (1.41-4.54) | | 4.34 (1.72-10.92) | | 1.70 (0.77-3.80) | |
| Q3 | 1.65 (0.95-2.88) | | 3.03 (1.19-7.75) | | 1.17 (0.57-2.40) | |
| Q4 | 2.37 (1.35-4.16) | 0.0077 | 3.57 (1.44-8.84) | 0.0147 | 2.00 (0.94-4.24) | 0.1330 |
| 9p | | | | | | |
| By median | 1.92 (1.26-2.92) | 0.0022 | 4.59 (2.29-9.20) | <0.0001 | 1.07 (0.61-1.88) | 0.8262 |
| By quartiles | | | | | | |
| Q2 | 0.94 (0.55-1.62) | | 1.22 (0.46-3.21) | | 1.03 (0.50-2.12) | |
| Q3 | 1.82 (1.01-3.28) | | 7.18 (2.48-20.79) | | 0.83 (0.38-1.80) | |
| Q4 | 1.98 (1.09-3.58) | 0.0052 | 4.29 (1.53-11.99) | 0.0002 | 1.45 (0.65-3.16) | 0.5411 |
| 12p | | | | | | |
| By median | 1.25 (0.83-1.87) | 0.2863 | 2.01 (1.06-3.84) | 0.0334 | 0.86 (0.50-1.49) | 0.5944 |
| By quartiles | | | | | | |
| Q2 | 1.21 (0.70-2.11) | | 1.31 (0.54-3.18) | | 1.10 (0.51-2.36) | |
| Q3 | 1.28 (0.73-2.24) | | 2.29 (0.91-5.74) | | 0.82 (0.38-1.75) | |
| Q4 | 1.46 (0.83-2.58) | 0.1907 | 2.25 (0.94-5.40) | 0.0366 | 1.00 (0.46-2.20) | 0.8116 |
| 15p | | | | | | |
| By median | 1.18 (0.78-1.78) | 0.4311 | 3.06 (1.58-5.95) | 0.0010 | 0.58 (0.33-1.01) | 0.0536 |
| By quartiles | | | | | | |
| Q2 | 0.78 (0.45-1.37) | | 1.32 (0.50-3.45) | | 0.64 (0.30-1.36) | |
| Q3 | 0.91 (0.51-1.63) | | 2.56 (0.99-6.64) | | 0.48 (0.22-1.04) | |
| Q4 | 1.12 (0.63-1.99) | 0.6238 | 4.44 (1.70-11.60) | 0.0008 | 0.42 (0.19-0.95) | 0.0234 |
| 15q | | | | | | |
| By median | 1.58 (1.05-2.38) | 0.0295 | 2.79 (1.44-5.40) | 0.0023 | 1.16 (0.66-2.03) | 0.6176 |
| By quartiles | | | | | | |
| Q2 | 1.07 (0.62-1.84) | | 1.09 (0.42-2.83) | | 0.90 (0.44-1.80) | |
| Q3 | 1.62 (0.91-2.88) | | 2.91 (1.17-7.27) | | 1.12 (0.51-2.48) | |
| Q4 | 1.65 (0.92-2.95) | 0.0418 | 2.89 (1.17-7.16) | 0.0053 | 1.05 (0.46-2.38) | 0.7789 |

*p-for-trend.
Bold p-values are significant at <0.01 level.
ORs were adjusted for age, race, education, household income, physical activity in teens, smoking status, alcohol use, family history of cancer and history of pregnancy.

TABLE 6
Comparing the Average Coefficient Variation (CV) of 46 Telomeres Between Cases and Controls

| | All subjects | | | Pre-menopausal women | | | Post-menopausal women | | |
|---|---|---|---|---|---|---|---|---|---|
| | cases | controls | p- | cases | controls | | cases | controls | p- |
| Type of telomeres | N = 204 | N = 236 | value† | N = 89 | N = 96 | p-value† | N = 110 | N = 132 | value† |
| Short version (S) | 64.52 | 63.16 | 0.0020 | 64.90 | 62.21 | 6.48 × 10⁻⁷ | 64.49 | 64.17 | 0.4537 |
| Long Version (L) | 44.22 | 43.36 | 0.0015 | 44.46 | 42.59 | 6.77 × 10⁻⁸ | 44.14 | 44.08 | 0.8903 |
| Combined (S + L) | 43.39 | 42.60 | 0.0025 | 43.67 | 41.90 | 1.50 × 10⁻⁷ | 43.30 | 43.29 | 0.9695 |

CV is expressed as %.
†p-values were from t-test.

TABLE 7
Case-Control comparison of Mean Coefficient of Variation (CV) of Telomere Length

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p-value† | N = 89 | N = 96 | p-value† | N = 110 | N = 132 | p-value† |
| 1p-AL* | 43.78 | 42.42 | 0.1076 | 45.05 | 41.23 | 0.0054 | 42.88 | 43.36 | 0.6780 |
| 5q-AS\hat{ } | 65.14 | 61.34 | 0.0073 | 66.14 | 59.95 | 0.0053 | 64.56 | 62.31 | 0.2271 |
| 8q-AS | 70.90 | 66.25 | 0.0051 | 71.67 | 65.94 | 0.0423 | 70.84 | 66.86 | 0.0569 |
| 12p-AL | 44.91 | 42.76 | 0.0174 | 45.96 | 41.45 | 0.0037 | 44.07 | 43.59 | 0.5355 |
| 15p-AS | 65.11 | 64.77 | 0.8731 | 66.42 | 61.26 | 0.0085 | 64.07 | 67.73 | 0.0606 |
| 18p-AS | 60.87 | 59.28 | 0.2380 | 61.93 | 56.85 | 0.0048 | 60.37 | 61.42 | 0.5972 |
| 18p-AL | 43.81 | 43.32 | 0.1121 | 44.41 | 40.42 | 0.0049 | 43.40 | 43.86 | 0.7310 |
| 19q-AL | 44.76 | 44.12 | 0.5118 | 46.29 | 42.82 | 0.0097 | 43.66 | 45.24 | 0.2115 |
| 21p-AS | 65.26 | 67.43 | 0.1002 | 66.47 | 66.25 | 0.9606 | 64.04 | 69.02 | 0.0074 |
| 21p-AL | 45.88 | 46.71 | 0.3556 | 46.94 | 45.07 | 0.1425 | 44.75 | 48.26 | 0.0090 |

*AL = allelically long version.
\hat{ }AS = allelically short version.
†Wilcoxon rank sum test was used for 5q-AS, 8q-AS, 12p-AL and 21p-AS, t-test was used for the other 6 telomeres.
CV is expressed as %.

TABLE 8
Logistic Regression Examining the Association between Telomere Length
Variation (TLV) and Breast Cancer Risk

| | | | Pre-menopausal | | Post-menopausal | |
|---|---|---|---|---|---|---|
| Chromosome | All subjects N = 440 | | women N = 185 | | women N = 242 | |
| arm | OR (95% CI) | p | OR (95% CI) | p | OR (95% CI) | p |
| 1p-AL | | | | | | |
| By median | 1.46 (0.97-2.19) | 0.0687 | 2.58 (1.33-5.02) | 0.0052 | 0.99 (0.57-1.71) | 0.9616 |
| By quartiles | | | | | | |
| Q2 | 0.72 (0.42-1.25) | | 0.74 (0.31-1.76) | | 0.62 (0.29-1.31) | |
| Q3 | 1.13 (0.63-2.03) | | 2.46 (0.94-6.47) | | 0.65 (0.30-1.43) | |
| Q4 | 1.33 (0.74-2.39) | 0.1624 | 1.99 (0.79-5.02) | 0.0313 | 0.90 (0.40-2.02) | 0.8275 |
| 5q-AS | | | | | | |
| By median | 1.26 (0.84-1.90) | 0.2634 | 1.78 (0.93-3.40) | 0.0798 | 1.01 (0.58-1.75) | 0.9800 |
| By quartiles | | | | | | |
| Q2 | 1.55 (0.89-2.71) | | 1.91 (0.75-4.87) | | 1.38 (0.66-2.89) | |
| Q3 | 1.22 (0.71-2.10) | | 1.71 (0.73-4.02) | | 0.95 (0.46-1.98) | |
| Q4 | 2.08 (1.15-3.76) | 0.0418 | 3.91 (1.47-10.42) | 0.0121 | 1.49 (0.67-3.31) | 0.5367 |
| 8q-AS | | | | | | |
| By median | 1.75 (1.15-2.65) | 0.0088 | 1.76 (0.93-3.32) | 0.0814 | 1.92 (1.07-3.43) | 0.0279 |
| By quartiles | | | | | | |
| Q2 | 0.76 (0.44-1.28) | | 1.15 (0.49-2.71) | | 0.52 (0.25-1.06) | |
| Q3 | 1.74 (0.93-3.28) | | 2.44 (0.93-6.41) | | 1.49 (0.62-3.56) | |
| Q4 | 1.32 (0.74-2.37) | 0.0824 | 1.54 (0.62-3.86) | 0.1707 | 1.25 (0.56-2.80) | 0.2192 |
| 12p-AL | | | | | | |
| By median | 1.56 (1.04-2.34) | 0.0331 | 2.34 (1.21-4.53) | 0.0115 | 1.17 (0.68-2.02) | 0.5803 |
| By quartiles | | | | | | |
| Q2 | 0.97 (0.56-1.68) | | 0.65 (0.26-1.63) | | 1.10 (0.52-2.32) | |
| Q3 | 1.44 (0.82-2.52) | | 2.12 (0.87-5.19) | | 1.09 (0.50-2.37) | |
| Q4 | 1.65 (0.92-2.95) | 0.0461 | 1.85 (0.75-4.55) | 0.0499 | 1.40 (0.62-3.15) | 0.4554 |
| 15p-AS | | | | | | |
| By median | 0.78 (0.52-1.17) | 0.2359 | 1.57 (0.83-2.98) | 0.1696 | 0.47 (0.27-0.82) | 0.0085 |
| By quartiles | | | | | | |
| Q2 | 0.94 (0.52-1.69) | | 1.50 (0.58-3.92) | | 0.67 (0.31-1.53) | |
| Q3 | 0.69 (0.39-1.20) | | 1.41 (0.58-3.44) | | 0.43 (0.19-0.94) | |
| Q4 | 0.85 (0.48-1.51) | 0.3807 | 3.10 (1.15-8.34) | 0.0389 | 0.37 (0.17-0.80) | 0.0061 |
| 18p-AS | | | | | | |
| By median | 1.33 (0.88-1.99) | 0.1725 | 2.40 (1.26-4.60) | 0.0080 | 0.83 (0.48-1.44) | 0.4985 |
| By quartiles | | | | | | |
| Q2 | 1.13 (0.64-1.97) | | 1.58 (0.65-3.86) | | 1.00 (0.47-2.15) | |
| Q3 | 1.18 (0.68-2.05) | | 2.42 (0.94-6.20) | | 0.72 (0.35-1.51) | |
| Q4 | 1.80 (1.00-3.25) | 0.0642 | 4.27 (1.5-11.58) | 0.0026 | 1.03 (0.47-2.25) | 0.7911 |
| 18p-AL | | | | | | |
| By median | 0.99 (0.66-1.48) | 0.9516 | 1.70 (0.89-3.22) | 0.1066 | 0.66 (0.38-1.15) | 0.1394 |
| By quartiles | | | | | | |
| Q2 | 0.92 (0.52-1.63) | | 1.12 (0.44-2.87) | | 0.91 (0.42-1.95) | |
| Q3 | 0.77 (0.44-1.35) | | 1.22 (0.48-3.14) | | 0.56 (0.27-1.16) | |
| Q4 | 1.36 (0.75-2.47) | 0.5065 | 2.90 (1.08-7.83) | 0.0355 | 0.91 (0.41-2.04) | 0.4543 |
| 19q-AL | | | | | | |
| By median | 1.10 (0.73-1.65) | 0.6472 | 2.54 (1.32-4.89) | 0.0054 | 0.53 (0.30-0.93) | 0.0274 |
| By quartiles | | | | | | |
| Q2 | 0.79 (0.45-1.38) | | 0.87 (0.36-2.09) | | 0.74 (0.34-1.64) | |
| Q3 | 1.08 (0.60-1.94) | | 2.82 (1.04-7.64) | | 0.52 (0.23-1.16) | |
| Q4 | 0.89 (0.50-1.56) | 0.9500 | 2.08 (0.85-5.09) | 0.0248 | 0.40 (0.18-0.88) | 0.0161 |
| 21p-AS | | | | | | |
| By median | 0.75 (0.50-1.13) | 0.1642 | 1.07 (0.56-2.04) | 0.8349 | 0.54 (0.31-0.93) | 0.0268 |
| By quartiles | | | | | | |
| Q2 | 0.95 (0.53-1.71) | | 1.22 (0.49-3.02) | | 0.90 (0.40-2.01) | |
| Q3 | 0.76 (0.43-1.36) | | 1.16 (0.45-2.98) | | 0.57 (0.26-1.23) | |
| Q4 | 0.70 (0.40-1.25) | 0.1652 | 1.22 (0.49-3.03) | 0.7212 | 0.45 (0.20-0.98) | 0.0240 |
| 21p-AL | | | | | | |
| By median | 0.70 (0.46-1.05) | 0.0806 | 1.20 (0.64-2.27) | 0.5729 | 0.44 (0.25-0.77) | 0.0042 |
| By quartiles | | | | | | |
| Q2 | 1.13 (0.61-2.07) | | 1.45 (0.54-3.93) | | 1.10 (0.47-2.55) | |
| Q3 | 0.71 (0.40-1.26) | | 1.08 (0.43-2.75) | | 0.56 (0.26-1..22) | |
| Q4 | 0.77 (0.43-1.38) | 0.1815 | 2.22 (0.83-5.93) | 0.1905 | 0.40 (0.18-0.81) | 0.0045 |

*p-for-trend.
Bold p-values are significant at <0.01 level.
ORs were adjusted for age, race, education, household income, physical activity in teens, smoking status, alcohol use, family history of cancer and history of pregnancy.
TLV is the coefficient variation (CV) of telomere length in 15 cells that were from the same individual.

TABLE 9
Case-Control Comparison of Mean Relative Telomere Length (RTL\hat{ })

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p† | N = 89 | N = 96 | p† | N = 110 | N = 132 | p† |

Short version of homologous telomeres

| | | | | | | | | | |
|---|---|---|---|---|---|---|---|---|---|
| 1p | 0.749 | 0.777 | 0.0432 | 0.744 | 0.805 | 0.0027 | 0.751 | 0.759 | 0.6687 |
| 1q | 0.773 | 0.778 | 0.7450 | 0.761 | 0.787 | 0.2623 | 0.780 | 0.770 | 0.6174 |
| 2p | 0.819 | 0.812 | 0.6207 | 0.825 | 0.830 | 0.8251 | 0.819 | 0.796 | 0.2522 |
| 2q | 0.692 | 0.707 | 0.2188 | 0.696 | 0.711 | 0.3823 | 0.687 | 0.703 | 0.3842 |
| 3p | 0.861 | 0.878 | 0.2829 | 0.863 | 0.879 | 0.5319 | 0.860 | 0.879 | 0.3825 |
| 3q | 0.752 | 0.752 | 0.9970 | 0.745 | 0.749 | 0.8398 | 0.755 | 0.750 | 0.7727 |
| 4p | 0.757 | 0.768 | 0.3967 | 0.759 | 0.791 | 0.0943 | 0.758 | 0.750 | 0.6999 |
| 4q | 0.837 | 0.860 | 0.1430 | 0.828 | 0.853 | 0.2843 | 0.843 | 0.861 | 0.3880 |
| 5p | 0.828 | 0.810 | 0.2664 | 0.839 | 0.807 | 0.1633 | 0.818 | 0.805 | 0.5354 |
| 5q | 0.707 | 0.741 | 0.0109 | 0.701 | 0.738 | 0.0609 | 0.711 | 0.745 | 0.0723 |
| 6p | 0.711 | 0.714 | 0.8081 | 0.691 | 0.713 | 0.2983 | 0.721 | 0.709 | 0.4974 |
| 6q | 0.779 | 0.794 | 0.3215 | 0.798 | 0.804 | 0.8083 | 0.765 | 0.789 | 0.2076 |
| 7p | 0.702 | 0.705 | 0.8462 | 0.715 | 0.721 | 0.7689 | 0.692 | 0.693 | 0.9490 |
| 7q | 0.780 | 0.800 | 0.1918 | 0.786 | 0.789 | 0.8932 | 0.775 | 0.801 | 0.1778 |
| 8p | 0.782 | 0.776 | 0.6847 | 0.803 | 0.777 | 0.2536 | 0.763 | 0.777 | 0.4329 |
| 8q | 0.650 | 0.676 | 0.0414 | 0.660 | 0.678 | 0.3838 | 0.638 | 0.678 | 0.0263 |
| 9p | 0.663 | 0.692 | 0.0239 | 0.665 | 0.719 | 0.0069 | 0.660 | 0.673 | 0.4449 |
| 9q | 0.645 | 0.640 | 0.6731 | 0.643 | 0.632 | 0.4798 | 0.649 | 0.644 | 0.8103 |
| 10p | 0.769 | 0.762 | 0.6309 | 0.770 | 0.746 | 0.1987 | 0.758 | 0.774 | 0.3728 |
| 10q | 0.726 | 0.725 | 0.9586 | 0.713 | 0.721 | 0.7172 | 0.738 | 0.730 | 0.6724 |
| 11p | 0.712 | 0.707 | 0.7095 | 0.717 | 0.702 | 0.4761 | 0.705 | 0.704 | 0.9725 |
| 11q | 0.715 | 0.721 | 0.5972 | 0.712 | 0.701 | 0.5536 | 0.720 | 0.738 | 0.3159 |
| 12p | 0.638 | 0.656 | 0.1413 | 0.621 | 0.667 | 0.0146 | 0.653 | 0.648 | 0.7462 |
| 12q | 0.740 | 0.748 | 0.5735 | 0.736 | 0.768 | 0.0995 | 0.742 | 0.731 | 0.5719 |
| 13p | 0.720 | 0.706 | 0.3322 | 0.743 | 0.702 | 0.0633 | 0.705 | 0.710 | 0.8226 |
| 13q | 0.771 | 0.783 | 0.4168 | 0.767 | 0.790 | 0.3312 | 0.776 | 0.779 | 0.8589 |
| 14p | 0.721 | 0.711 | 0.4879 | 0.728 | 0.716 | 0.6015 | 0.708 | 0.709 | 0.9395 |
| 14q | 0.721 | 0.714 | 0.6078 | 0.720 | 0.729 | 0.6778 | 0.723 | 0.703 | 0.2811 |
| 15p | 0.700 | 0.697 | 0.7968 | 0.682 | 0.740 | 0.0073 | 0.713 | 0.664 | 0.0078 |
| 15q | 0.670 | 0.682 | 0.2992 | 0.670 | 0.706 | 0.0591 | 0.669 | 0.665 | 0.8421 |
| 16p | 0.626 | 0.631 | 0.6975 | 0.627 | 0.627 | 0.9869 | 0.622 | 0.631 | 0.5676 |
| 16q | 0.646 | 0.660 | 0.2198 | 0.634 | 0.673 | 0.0314 | 0.652 | 0.650 | 0.8772 |
| 17p | 0.590 | 0.603 | 0.2169 | 0.610 | 0.615 | 0.7768 | 0.576 | 0.596 | 0.1455 |
| 17q | 0.611 | 0.603 | 0.4947 | 0.608 | 0.604 | 0.7911 | 0.609 | 0.602 | 0.6577 |
| 18p | 0.718 | 0.726 | 0.5698 | 0.709 | 0.743 | 0.1142 | 0.724 | 0.715 | 0.6462 |
| 18q | 0.710 | 0.720 | 0.4812 | 0.689 | 0.718 | 0.1746 | 0.725 | 0.716 | 0.6219 |
| 19p | 0.583 | 0.587 | 0.7042 | 0.595 | 0.591 | 0.8143 | 0.570 | 0.581 | 0.4629 |
| 19q | 0.622 | 0.629 | 0.5405 | 0.606 | 0.625 | 0.3208 | 0.630 | 0.631 | 0.9635 |
| 20p | 0.628 | 0.635 | 0.5656 | 0.631 | 0.637 | 0.7191 | 0.625 | 0.633 | 0.5789 |
| 20q | 0.572 | 0.577 | 0.6136 | 0.573 | 0.570 | 0.8473 | 0.571 | 0.583 | 0.4073 |
| 21p | 0.678 | 0.653 | 0.0713 | 0.679 | 0.671 | 0.6857 | 0.679 | 0.636 | 0.0273 |
| 21q | 0.594 | 0.595 | 0.9477 | 0.594 | 0.595 | 0.9757 | 0.591 | 0.592 | 0.9214 |
| 22p | 0.675 | 0.686 | 0.4403 | 0.656 | 0.674 | 0.4151 | 0.693 | 0.686 | 0.6917 |
| 22q | 0.576 | 0.580 | 0.6673 | 0.564 | 0.583 | 0.2409 | 0.583 | 0.579 | 0.7821 |
| Xp | 0.783 | 0.802 | 0.2193 | 0.759 | 0.834 | 0.0008 | 0.780 | 0.778 | 0.2852 |
| Xq | 0.718 | 0.713 | 0.6992 | 0.718 | 0.728 | 0.6179 | 0.718 | 0.703 | 0.4206 |
| 1p | 1.558 | 1.594 | 0.1130 | 1.540 | 1.607 | 0.0476 | 1.574 | 1.595 | 0.5076 |
| 1q | 1.584 | 1.586 | 0.9305 | 1.570 | 1.583 | 0.6748 | 1.599 | 1.588 | 0.7027 |
| 2p | 1.652 | 1.656 | 0.8485 | 1.655 | 1.650 | 0.8636 | 1.657 | 1.663 | 0.8362 |
| 2q | 1.439 | 1.439 | 0.9889 | 1.467 | 1.422 | 0.1108 | 1.422 | 1.447 | 0.3470 |
| 3p | 1.751 | 1.754 | 0.9002 | 1.751 | 1.738 | 0.7133 | 1.750 | 1.768 | 0.5239 |
| 3q | 1.538 | 1.500 | 0.0397 | 1.553 | 1.486 | 0.0150 | 1.530 | 1.502 | 0.3006 |
| 4p | 1.545 | 1.559 | 0.4797 | 1.565 | 1.560 | 0.8812 | 1.524 | 1.557 | 0.1996 |
| 4q | 1.706 | 1.720 | 0.5342 | 1.682 | 1.695 | 0.6953 | 1.726 | 1.734 | 0.7906 |
| 5p | 1.712 | 1.690 | 0.3525 | 1.707 | 1.656 | 0.1565 | 1.717 | 1.711 | 0.8499 |
| 5q | 1.500 | 1.503 | 0.8568 | 1.512 | 1.465 | 0.1215 | 1.494 | 1.533 | 0.1666 |
| 6p | 1.486 | 1.478 | 0.6886 | 1.472 | 1.482 | 0.7660 | 1.491 | 1.472 | 0.4690 |
| 6q | 1.623 | 1.613 | 0.6289 | 1.647 | 1.603 | 0.1757 | 1.609 | 1.617 | 0.7564 |
| 7p | 1.486 | 1.492 | 0.7771 | 1.497 | 1.480 | 0.6127 | 1.483 | 1.506 | 0.3785 |
| 7q | 1.626 | 1.630 | 0.8233 | 1.646 | 1.590 | 0.0860 | 1.609 | 1.661 | 0.0621 |
| 8p | 1.625 | 1.596 | 0.2010 | 1.620 | 1.604 | 0.6214 | 1.628 | 1.597 | 0.3064 |
| 8q | 1.454 | 1.444 | 0.6300 | 1.480 | 1.433 | 0.1381 | 1.434 | 1.459 | 0.3865 |
| 9p | 1.438 | 1.427 | 0.5605 | 1.450 | 1.455 | 0.8674 | 1.432 | 1.411 | 0.4235 |
| 9q | 1.343 | 1.338 | 0.7943 | 1.323 | 1.311 | 0.6818 | 1.357 | 1.349 | 0.7370 |
| 10p | 1.567 | 1.561 | 0.7671 | 1.576 | 1.536 | 0.1814 | 1.551 | 1.581 | 0.2758 |
| 10q | 1.523 | 1.507 | 0.4516 | 1.519 | 1.486 | 0.2821 | 1.529 | 1.521 | 0.7815 |
| 11p | 1.468 | 1.476 | 0.7278 | 1.499 | 1.484 | 0.6518 | 1.445 | 1.465 | 0.4936 |
| 11q | 1.479 | 1.463 | 0.4166 | 1.469 | 1.439 | 0.2736 | 1.494 | 1.489 | 0.8556 |
| 12p | 1.368 | 1.338 | 0.0770 | 1.364 | 1.348 | 0.5541 | 1.376 | 1.335 | 0.0723 |
| 12q | 1.599 | 1.595 | 0.8535 | 1.623 | 1.602 | 0.5009 | 1.581 | 1.585 | 0.8941 |
| 13p | 1.522 | 1.517 | 0.8026 | 1.507 | 1.519 | 0.6806 | 1.541 | 1.523 | 0.5660 |
| 13q | 1.619 | 1.602 | 0.4375 | 1.620 | 1.591 | 0.3770 | 1.621 | 1.609 | 0.7071 |
| 14p | 1.542 | 1.528 | 0.5462 | 1.556 | 1.525 | 0.4115 | 1.526 | 1.534 | 0.7942 |
| 14q | 1.498 | 1.502 | 0.8396 | 1.490 | 1.483 | 0.8021 | 1.508 | 1.520 | 0.6663 |
| 15p | 1.525 | 1.489 | 0.1193 | 1.521 | 1.497 | 0.4909 | 1.529 | 1.485 | 0.1700 |
| 15q | 1.428 | 1.392 | 0.0437 | 1.432 | 1.395 | 0.1633 | 1.427 | 1.394 | 0.1987 |
| 16p | 1.323 | 1.347 | 0.2433 | 1.321 | 1.329 | 0.7978 | 1.322 | 1.356 | 0.2451 |
| 16q | 1.371 | 1.374 | 0.8659 | 1.355 | 1.370 | 0.6228 | 1.381 | 1.370 | 0.6608 |
| 17p | 1.278 | 1.264 | 0.3967 | 1.304 | 1.262 | 0.1210 | 1.264 | 1.270 | 0.7949 |
| 17q | 1.287 | 1.281 | 0.7476 | 1.284 | 1.289 | 0.8712 | 1.288 | 1.275 | 0.5968 |
| 18p | 1.432 | 1.427 | 0.7862 | 1.415 | 1.453 | 0.1838 | 1.444 | 1.412 | 0.2018 |
| 18q | 1.491 | 1.474 | 0.4166 | 1.476 | 1.458 | 0.6247 | 1.503 | 1.482 | 0.4723 |
| 19p | 1.247 | 1.245 | 0.9397 | 1.268 | 1.265 | 0.9183 | 1.233 | 1.230 | 0.9170 |
| 19q | 1.329 | 1.301 | 0.1137 | 1.319 | 1.295 | 0.4053 | 1.337 | 1.308 | 0.1844 |
| 20p | 1.355 | 1.348 | 0.6971 | 1.352 | 1.349 | 0.9081 | 1.354 | 1.345 | 0.7389 |
| 20q | 1.230 | 1.211 | 0.2732 | 1.246 | 1.198 | 0.0665 | 1.220 | 1.226 | 0.7742 |
| 21p | 1.472 | 1.415 | 0.0106 | 1.467 | 1.406 | 0.0723 | 1.483 | 1.421 | 0.0466 |
| 21q | 1.264 | 1.245 | 0.2524 | 1.263 | 1.244 | 0.4407 | 1.264 | 1.247 | 0.4719 |
| 22p | 1.454 | 1.474 | 0.3597 | 1.461 | 1.468 | 0.8557 | 1.455 | 1.473 | 0.5368 |
| 22q | 1.229 | 1.237 | 0.6577 | 1.211 | 1.229 | 0.4840 | 1.242 | 1.247 | 0.8456 |
| Xp | 1.675 | 1.667 | 0.7568 | 1.657 | 1.693 | 0.3217 | 1.692 | 1.656 | 0.2745 |
| Xq | 1.471 | 1.462 | 0.6379 | 1.483 | 1.454 | 0.2846 | 1.465 | 1.474 | 0.7373 |

\hat{ }RTL was defined as the percent of arm-specific telomere fluorescent intensity unites (FIU) divided by total telomere FIU of 92 telomeres.
†all p-values were based on Student t-test.
Bold p-values were significant at <0.01 level.

TABLE 10
Case-Control Comparison of Mean Homologous Telomere Length Differences (HTLD)

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p† | N = 89 | N = 96 | p† | N = 110 | N = 132 | p† |
| 1p | 36.76 | 36.28 | 0.4977 | 36.43 | 34.83 | 0.1483 | 37.30 | 37.50 | 0.8336 |
| 1q | 36.81 | 36.36 | 0.5423 | 37.21 | 35.50 | 0.1215 | 36.85 | 37.14 | 0.7852 |
| 2p | 36.24 | 36.40 | 0.8255 | 35.86 | 34.92 | 0.3768 | 36.52 | 37.68 | 0.2336 |
| 2q | 37.06 | 36.28 | 0.2428 | 37.32 | 35.59 | 0.0790 | 37.24 | 36.86 | 0.6821 |
| 3p | 36.21 | 35.33 | 0.2051 | 36.13 | 35.30 | 0.4352 | 36.29 | 35.40 | 0.3446 |
| 3q | 36.21 | 35.18 | 0.1223 | 36.88 | 34.89 | 0.0553 | 35.87 | 35.43 | 0.6310 |
| 4p | 36.51 | 36.10 | 0.5403 | 37.06 | 34.83 | 0.0231 | 35.88 | 37.17 | 0.1656 |
| 4q | 36.28 | 35.02 | 0.0782 | 36.29 | 34.56 | 0.1134 | 36.38 | 35.39 | 0.3174 |
| 5p | 36.88 | 37.03 | 0.8358 | 36.21 | 36.33 | 0.9109 | 37.46 | 37.76 | 0.7680 |
| 5q | 38.45 | 36.21 | 0.0013 | 39.39 | 35.60 | 0.0006 | 37.93 | 36.61 | 0.1509 |
| 6p | 37.77 | 36.82 | 0.2181 | 38.73 | 36.81 | 0.1214 | 37.12 | 37.06 | 0.9517 |
| 6q | 37.48 | 36.19 | 0.0908 | 36.80 | 35.18 | 0.1674 | 38.13 | 36.76 | 0.1803 |
| 7p | 37.63 | 37.55 | 0.9123 | 37.04 | 36.36 | 0.5393 | 38.32 | 38.53 | 0.8267 |
| 7q | 37.33 | 36.38 | 0.2079 | 37.66 | 35.82 | 0.1018 | 37.09 | 37.10 | 0.9896 |
| 8p | 37.06 | 36.78 | 0.6739 | 35.84 | 36.96 | 0.2828 | 38.11 | 36.82 | 0.1420 |
| 8q | 40.68 | 38.48 | 0.0036 | 40.93 | 38.00 | 0.0077 | 40.80 | 38.90 | 0.0771 |
| 9p* | 38.91 | 37.14 | 0.0169 | 39.10 | 35.90 | 0.0005 | 38.95 | 38.11 | 0.6494 |
| 9q | 36.89 | 37.22 | 0.6576 | 6.34 | 36.99 | 0.543 | 37.22 | 3734 | 0.9118 |
| 10p | 36.22 | 36.39 | 0.8100 | 36.35 | 36.62 | 0.7965 | 36.46 | 36.39 | 0.9461 |
| 10q | 37.39 | 37.25 | 0.8512 | 37.91 | 36.93 | 0.4078 | 36.94 | 37.43 | 0.6193 |
| 11p | 36.69 | 36.98 | 0.6725 | 37.59 | 37.48 | 0.9130 | 36.21 | 36.89 | 0.4821 |
| 11q | 36.95 | 36.36 | 0.3937 | 36.96 | 36.81 | 0.8871 | 37.00 | 36.25 | 0.4278 |
| 12p | 38.80 | 36.86 | 0.0081 | 39.76 | 36.55 | 0.0058 | 38.18 | 37.30 | 0.3627 |
| 12q | 38.80 | 38.12 | 0.3464 | 39.59 | 37.44 | 0.0425 | 38.44 | 38.70 | 0.7988 |
| 13p | 38.01 | 38.51 | 0.4929 | 36.17 | 38.99 | 0.0124 | 39.51 | 38.40 | 0.2659 |
| 13q | 37.61 | 36.45 | 0.1077 | 38.21 | 35.86 | 0.0480 | 37.19 | 36.90 | 0.7532 |
| 14p | 38.63 | 38.56 | 0.9202 | 38.89 | 38.20 | 0.5333 | 38.72 | 38.90 | 0.8550 |
| 14q | 37.31 | 37.69 | 0.5887 | 37.38 | 36.20 | 0.2735 | 37.31 | 38.87 | 0.1029 |
| 15p | 38.95 | 38.19 | 0.3038 | 40.14 | 35.87 | 0.0001 | 38.08 | 40.00 | 0.0533 |
| 15q | 38.13 | 36.55 | 0.0295 | 38.16 | 34.87 | 0.0034 | 38.20 | 37.99 | 0.8240 |
| 16p | 38.17 | 37.98 | 0.7831 | 38.12 | 37.95 | 0.8701 | 38.41 | 38.07 | 0.7142 |
| 16q | 37.68 | 36.93 | 0.2885 | 37.94 | 36.31 | 0.1357 | 37.59 | 37.30 | 0.7586 |
| 17p | 39.63 | 37.71 | 0.0038 | 39.27 | 37.49 | 0.0866 | 39.82 | 37.90 | 0.0346 |
| 17q | 38.01 | 38.19 | 0.8107 | 38.15 | 38.62 | 0.6774 | 38.16 | 37.84 | 0.7460 |
| 18p | 34.98 | 34.63 | 0.6228 | 35.17 | 34.06 | 0.2923 | 34.90 | 35.18 | 0.7841 |
| 18q | 37.83 | 36.54 | 0.0789 | 38.79 | 36.24 | 0.0249 | 37.28 | 37.05 | 0.8172 |
| 19p | 38.16 | 37.86 | 0.6610 | 38.06 | 38.08 | 0.9824 | 38.55 | 37.83 | 0.4400 |
| 19q | 38.71 | 37.07 | 0.0308 | 39.44 | 37.31 | 0.0557 | 38.43 | 37.06 | 0.1963 |
| 20p | 38.87 | 38.01 | 0.2295 | 38.42 | 37.62 | 0.4612 | 39.12 | 38.27 | 0.3467 |
| 20q | 38.61 | 37.30 | 0.0648 | 39.30 | 36.96 | 0.0362 | 38.05 | 37.67 | 0.6911 |
| 21p | 38.68 | 38.87 | 0.7816 | 38.37 | 37.46 | 0.3888 | 38.95 | 40.22 | 0.1951 |
| 21q | 38.32 | 37.52 | 0.2763 | 38.03 | 37.46 | 0.5981 | 38.72 | 37.75 | 0.3421 |
| 22p | 39.04 | 38.96 | 0.9072 | 40.50 | 39.19 | 0.2606 | 37.95 | 39.16 | 0.2106 |
| 22q | 38.44 | 38.22 | 0.7650 | 38.76 | 37.58 | 0.2785 | 38.32 | 38.86 | 0.5914 |
| Xp | 38.27 | 37.16 | 0.1280 | 39.37 | 36.40 | 0.0076 | 37.62 | 37.95 | 0.7445 |
| Xq | 36.65 | 36.22 | 0.5363 | 36.88 | 35.42 | 0.1641 | 36.62 | 37.04 | 0.6720 |

HTLD was defined as the percent of (homologous long RTL − homologous short RTL) divided by (homologous long RTL + homologous short RTL)
†all p-values were based on Student t-test except for 9p
*Wilcoxon ranks um test was used
Bold p-values were significant at <0.01 level

### III. Discussion

This data shows that, after adjustment for known breast cancer risk factors, shorter telomere lengths on chromosome Xp and 15p were significantly associated with an increased risk of breast cancer in pre-menopausal women. These data support the hypothesis that women who have telomere length deficiency on certain chromosome arms are at increased risk of breast cancer. The present study is the first study that examined the association between all 92 individual telomeres in the human genome and risk of breast cancer. The results provided new evidence that short telomere lengths on chromosomes Xp and 15p were significantly associated with breast cancer risk in pre-menopausal women. The data also revealed that telomere lengths between non-homologous telomeres were not correlated and are likely independent genetic events that may carry information of clinical importance for cancer patients.

Deficiencies in telomere health is particularly relevant to carcinogenesis because hyper-proliferative cancerous cells could lead to progressive telomere shortening, ultimately generating uncapped telomeres that fuse with each other leading to genomic instability that promotes malignant transformation. However, it has not been established if it is the shortest telomeres or the mean telomere length that triggers the telomere dysfunction-associated responses. The discoveries and results described herein support the former mechanism. The discoveries and results described herein are also consistent with the report that individual dysfunctional telomeres are recognized as DNA damage and a cellular response is triggered (Artandi, 2005). Crossing telomerase knockout mice having short telomeres with those having long telomeres revealed that loss of telomere function occurs preferentially on the shortest telomere and that the shortest telomeres, rather than the average telomere length, elicit a cellular response (Hemann, 2001). The discoveries and results described herein are also consistent with reports that chromosome arms carrying the shortest telomeres were more often found in telomere fusions leading to chromosomal instability (Der-Sarkissian, 2004; Soler, 2005) (Capper R et al 2007, 21:2495). The discoveries and results described herein are also consistent with reports that, in humans, chromosome specific telomere lengths are highly variable between chromosomal arms (Gilson, 2007; Lansdorp, 1996; Graakjaer, 2003; Martens, 1998). The chromosome arm-specific telomere length polymorphism disclosed herein indicates that chromosome arms bearing the shortest telomeres may predispose to the chromosome alterations and therefore have an impact on the evolution of tumors. Regardless of this mechanism, the disclosed results and discoveries show that certain measures of individual telomere length provide an indication of cancer risk.

The comprehensive approaches used in this example allowed for examination of the associations between telomere length variations and breast cancer risk. One of the main efforts of telomere maintenance is to provide homogenous protection for all the telomeres and to minimize the opportunity for induction of dysfunctional telomeres. It was realized that high degree telomere length variations represent a deficiency in telomere maintenance and are linked to cancer susceptibility. To demonstrate this, the association between homologous telomere length difference (HTLD) and breast cancer risk were examined. The data indicated that greater HTLDs on chromosome 9p, 15p and 15q were significantly associated with breast cancer risk in pre-menopausal women. These data indicated that telomere length variations between homologous telomeres can represent different phenotypes of telomere deficiency that is linked to breast cancer susceptibility. This example introduces homologous telomere length difference as a new phenotype of deficiencies in telomere maintenance and identified greater HTLDs on chromosome 9p, 15p and 15q as new risk factors for breast cancer in premenopausal women.

This example also revealed that mean telomere length variation of 46 chromosome arms in lymphocytes is significantly higher in cases than in controls (p=1.50×10⁻⁷) in pre-menopausal women (Table 6). Examining individual telomere length variation in lymphocytes identified telomere length variation on 18p showing suggestive association with breast cancer risk in premenopausal women (Table 7). These data provided further evidence that greater telomere length heterogeneity can contribute to an increased breast cancer risk in premenopausal women.

Telomere capping presents a unique challenge to a proliferative cell. Because telomerase is nonessential and its activity is undetectable in most normal human somatic cells, an alternative mechanism that contributes to telomere maintenance may play the key role for telomere homeostasis in normal somatic cells. There is a growing body of evidence that homologous recombination (HR) proteins and other proteins involved in DNA repair have a complex role in normal telomere biology (Sarthy J 2009, 29:3390; Wu Y 2008, 129:602; Zeng S 2009, 11:616; Opresko P L 2004, 14:763; Poulet A 2009, 28:641; Verdum R E 2006, 127:709; Wang R C 2004, 119:355). During DNA replication, telomeres cycle through what appears to be recognition of chromosome ends as DNA damage during specific phases of the cell cycle (Verdun R E, 2005, 20:551; Verdun R E, 2006, 127:709). While the action of telomere binding proteins inhibit end-to-end fusions (Bae and Baumann 2007, 26:323), the temporary recruitment of HR proteins to the telomeres in S phase and early G2 phases is necessary for the HR-assisted capping during G2 to restructure the chromosome terminus into a t-loop, preventing the recognition of chromosome ends as DSBs in G1 (Verdun R E, 2005, 20:551; Verdun R E, 2006, 127:709). Therefore, well controlled moderate telomere HR is beneficial to capping-related roles. In the absence of proper regulation, telomere HR could result in several deleterious consequences, including telomere rapid deletions, recombinational telomere elongation or immortalization via the alternative lengthening of telomeres (ALT) pathway (De Boeck G 2009, 217:327). ALT mechanism may arise via a loss of function in the complex controls over telomere capping, leading to a telomere-specific increases in HR (Jiang W Q 2005, 25:2708; Potts P R 2007, 14:581; Zhong Z H 2007, 282:29314).

A substantial number of human malignant tumors utilize ALT, a telomerase-independent telomere length maintenance mechanism. These include bone and soft tissue sarcomas, glioblastomas, and carcinomas of the lung, kidney, breast and ovary (Bryan tm 1997, 3:1271; Mehle c 1996, 13:161). ALT-mediated telomere length maintenance is characterized by highly heterogeneous telomeric DNA (Bryan tm 1995, 14:4240; Henson J D 2002, 21:598; Cesare A J 2004, 24:9948). ALT telomere length dynamics are complex, with both rapid elongation and rapid deletion events superimposed on a background of constant telomere attrition (Murnane J P 1994, 13:4953). Telomere FISH showed that within any ALT cell, there are telomeres that ranged from <2 kb to >50 kb in length, and often several chromosome ends lacking any telomere signal (Henson J D 2002, 21:598). Given the important role of HR in normal telomere biology, it is possible that dysregulation of HR-assisted telomere maintenance can result in increased telomere variations between individual telomeres in somatic cells, resulting in an increased risk of cancer. The observation herein that greater length variations between homologous telomeres and among somatic cells are associated with an increased risk of breast cancer in pre-menopausal women is consistent with this mechanism.

The data in this example indicates that the associations between telomere deficiencies and breast cancer risk in pre-menopausal women only involve a handful of chromosome arms (Xp, 9p, 15p, 15q and 18p). The reason why these chromosomal arms are associated with this cancer risk may be related to telomere-mediated dysregulation of genes that reside on those chromosome arms and that are involved in breast carcinogenesis. For example, telomere lengths are shown to be the critical players in regulating epigenetic modification of regional chromatin and these telomere-related epigenetic changes could result in epigenetic dysregulation of oncogenes and/or tumor suppressor genes (Benetti, 2007; Garcia-Cao, 2004; Vera, 2008). Deficiency in 9p telomeres could potentially affect the stability of chromosome 9p, where the CDKN2A locus (also known as INK4a/ARF locus) locates at 9p21. CDKN2A locus encodes two proteins, p16^{INK4a} and p14^{ARF}, that regulate 2 critical cell cycle regulatory pathways: the p53 pathway and the retinoblastoma pathway (Harris, 2005; Sherr, 1996; Sherr, 1998). Inactivation of CDKN2A locus removes an important barrier to tumor progression and 9p21 is a frequent target of inactivation by deletion or aberrant DNA methylation in a wide variety of human cancers (Kim, 2006 1158/id), including breast cancer (Ellsworth, 2007; Hwang, 2004; Tao, 2009; Esteller, 2001; Gorgoulis, 1998). Despite its importance in tumor suppression and considerable research, the cause of CDKN2A inactivation by deletion or aberrant promoter methylation is still unknown.

The data in this example indicated that the association between chromosome arm specific telomere deficiencies and breast cancer risk is restricted to pre-menopausal women. In post-menopausal women, there is a suggestive association between greater telomere length variation in somatic cells on chromosomes 15p and 21p and decreased breast cancer risk. However, it should be noted that none of the associations in post-menopausal women were statistically significant after considering Bonferroni correction for multiple comparisons.

Given that this is a case-control study, there could have been a theoretical concern is that telomere length in lymphocytes is affected by case status (reverse causality). However, reverse causality is not a plausible explanation for the results. Data by previous studies and current studies indicated that the mean overall telomere length of blood leucocytes in breast cancer patients was not significantly shorter than in healthy women controls (Zheng, 2009; De, 2009; Shen, 2007; Barwell, 2007), suggesting there is no significant shortening of blood leucocyte telomere length associated with having breast cancer. Although previous studies (Schroder, 2001; Yoon, 2007) suggested that chemotherapy and/or radiotherapy can induce telomere shortening in leucocytes, all the blood samples in this example were drawn before any chemotherapy and radiotherapy treatments. Thus reverse causality is not a plausible explanation for the results. This example is limited by its moderate sample size and is not powered to detect the small to moderate associations (i.e. OR<2.0).

In summary, this example revealed that short telomere length on chromosome Xp and 15p, greater length differences between homologous telomeres on chromosome 9p, 15p and 15q, and greater telomere length variation in lymphocytes on chromosome 18p were significantly associated with breast cancer risk in premenopausal women. These data provided first evidence that telomere deficiency (poor telomere health) on certain chromosome arms are linked to breast cancer susceptibility. As described elsewhere herein, these new discoveries have clinical application in detecting and assessing cancer risk. Telomere-related parameters can be used as a panel of blood-based biomarkers for breast cancer risk assessment, given their strong associations with breast cancer risk. Better risk assessment would improve the efficiency of both population-based preventive programs, such as screening mammography, as well as individual-based preventive strategies such as chemoprevention by targeting women who are at the greatest risk for breast cancer.

### Example 2

### Correlation Analysis of Lung Cancer Risk with Telomere Length, Telomere Length Variation, and Frequency of Extremely Short Telomeres

This example describes a genome-wide telomere association study to examine the associations between lengths of 92 telomeres in blood lymphocytes and lung cancer risk. The correlations discovered indicate roles of chromosomal telomeres in lung cancer susceptibility and provide the foundation of the disclosed methods.

The study involved 189 cases diagnosed with lung cancer and 205 disease-free controls. Table 11 shows the demographic characteristics of the study subjects.

TABLE 11
Demographic characteristics of study subjects

| | Cases | | Control | | |
|---|---|---|---|---|---|
| | N = 189 | | N = 206 | | p-value |
| Age, mean (SD) | 67.1 | (12.0) | 66.4 | (12.1) | 0.57 |
| Age distribution, N (%) | | | | | |
| 40-50 | 20 | (10.7) | 22 | (10.7) | |
| 51-60 | 32 | (17.1) | 38 | (18.5) | |
| 61-70 | 51 | (27.3) | 58 | (28.3) | |
| 71-80 | 63 | (33.7) | 65 | (31.7) | |
| ≧81 | 21 | (11.2) | 22 | (10.7) | 0.99 |
| Gender, N (%) | | | | | |
| Female | 99 | (52.4) | 111 | (53.9) | |
| Male | 90 | (47.6) | 95 | (46.1) | 0.77 |
| Race, N (%) | | | | | |
| African American | 67 | (35.5) | 78 | (37.9) | |
| Caucasian American | 122 | (64.5) | 128 | (62.1) | 0.62 |
| Smoking Status, N (%) | | | | | |
| Never | 11 | (5.8) | 78 | (37.9) | |
| Former | 75 | (39.7) | 95 | (46.1) | |
| Current | 103 | (54.5) | 33 | (16.0) | <0.001 |
| Pack-years, N (%) | | | | | |
| ≦20 | 36 | (20.3) | 61 | (48.0) | |
| 21-40 | 60 | (33.9) | 34 | (26.8) | |
| 41-60 | 47 | (26.7) | 22 | (17.3) | |
| >60 | 34 | (19.2) | 10 | (7.9) | <0.001 |
| Education, N (%) | | | | | |
| High school or less | 160 | (88.9) | 145 | (87.4) | |
| College or higher | 20 | (11.1) | 21 | (12.7) | 0.66 |
| Income, N (%) | | | | | |
| Less than 10K | 25 | (15.4) | 8 | (5.1) | |
| 10K-30K | 62 | (38.3) | 48 | (30.6) | |
| 30K-60K | 42 | (25.9) | 40 | (25.5) | |
| Greater than 60K | 33 | (20.4) | 61 | (38.9) | <0.001 |
| Marital Status, N (%) | | | | | |
| Single, never married | 10 | (5.3) | 13 | (6.3) | |
| Married or has a partner | 107 | (56.6) | 138 | (67.0) | |
| Divorced, separated, or | 72 | (38.1) | 55 | (26.7) | 0.053 |
| widowed | | | | | |
| Family History of Cancer, N | | | | | |
| (%) | | | | | |
| No | 43 | (22.8) | 47 | (22.8) | |
| Yes | 146 | (77.3) | 159 | (77.2) | 0.99 |
| BMI, mean (SD) | 26.0 | (5.0) | 26.6 | (5.1) | 0.24 |
| BMI, N (%) | | | | | |
| <20 | 12 | (6.4) | 14 | (6.8) | |
| 20-25 | 79 | (42.0) | 67 | (32.7) | |
| 25-30 | 61 | (32.5) | 80 | (39.0) | |
| ≧30 | 36 | (19.2) | 44 | (21.5) | 0.29 |
| Physical activity (hrs/week), | | | | | |
| N (%) | | | | | |
| <=7 | 61 | (32.6) | 55 | (26.7) | |
| 8-11 | 28 | (15.0) | 37 | (18.0) | |
| 13-21 | 43 | (23.0) | 63 | (30.6) | |
| >=22 | 55 | (29.4) | 51 | (24.8) | 0.21 |

Telomere lengths in cells from the cases were determined and analyzed generally as described herein. Significant correlations were found for telomere length variation (TLV) and frequency of extremely short telomeres in subjects 60 years or younger indicating a risk of lung cancer.

Table 12 shows a case-control comparison of telomere parameters. The data show significant correlations of telomere length variation and percent of short telomeres to the risk of lung cancer in subject 60 years of age and younger. In this analysis, short telomeres were defined as telomeres that were shorter than 10% of the average telomere length. The percent of short telomeres is equivalent to frequency of extremely short telomeres by converting the percent of short telomeres to the number of short telomeres divided by the total number of telomeres. Thus, for example, 3.3% of short telomeres is equivalent to a frequency of extremely short telomeres of 0.033.

TABLE 12
Case-control comparison of telomere parameters

| Telomere parameters | Cases mean (SD) | Controls mean (SD) | p-value |
|---|---|---|---|
| All subjects, N | 189 | 206 | |
| TL | 2417 (700) | 2410 (645) | 0.91 |
| TLV | 65.0 (6.2) | 65.0 (6.2) | 0.90 |
| % of short telomeres | 3.6 (1.6) | 3.7 (1.7) | 0.48 |
| AG_ratio | 1.3 (0.2) | 1.3 (0.1) | 0.28 |
| Age <= 60, N | 54 | 61 | |
| TL | 2388 (637) | 2530 (775) | 0.29 |
| TLV | 63.3 (7.2) | 60.2 (4.5) | 0.006 |
| % of short telomeres | 3.3 (1.7) | 2.4 (1.0) | <0.001 |
| AG_ratio | 1.2 (0.1) | 1.3 (0.1) | 0.044 |
| Age 61-74, N | 67 | 73 | |
| TL | 2678 (637) | 2618 (538) | 0.55 |
| TLV | 66.5 (5.6) | 68.5 (4.9) | 0.028 |
| % of short telomeres | 4.0 (1.7) | 4.6 (1.6) | 0.028 |
| AG_ratio | 1.3 (0.2) | 1.3 (0.2) | 0.23 |
| Age >= 75, N | 68 | 72 | |
| TL | 2183 (731) | 2096 (660) | 0.46 |
| TLV | 64.7 (5.6) | 65.7 (6.0) | 0.34 |
| % of short telomeres | 3.5 (1.4) | 4.0 (1.7) | 0.07 |
| AG_ratio | 1.2 (0.1) | 1.3 (0.1) | 0.12 |
| Male, N | 90 | 95 | |
| TL | 2377 (677) | 2440 (703) | 0.54 |
| TLV | 66.2 (5.9) | 65.3 (6.1) | 0.33 |
| % of short telomeres | 3.9 (1.6) | 3.8 (1.7) | 0.57 |
| AG_ratio | 1.3 (0.1) | 1.3 (0.1) | 1.00 |
| Female, N | 99 | 111 | |
| TL | 2453 (723) | 2383 (690) | 0.47 |
| TLV | 63.9 (6.3) | 64.8 (6.3) | 0.28 |
| % of short telomeres | 3.4 (1.6) | 3.7 (1.8) | 0.13 |
| AG_ratio | 1.3 (0.2) | 1.3 (0.1) | 0.15 |
| African American, N | 67 | 78 | |
| TL | 2621 (629) | 2504 (647) | 0.27 |
| TLV | 64.8 (6.1) | 65.3 (6.5) | 0.60 |
| % of short telomeres | 3.5 (1.7) | 3.9 (1.9) | 0.11 |
| AG_ratio | 1.3 (0.2) | 1.3 (0.1) | 0.56 |
| Caucasian, N | 122 | 128 | |
| TL | 2305 (720) | 2352 (718) | 0.61 |
| TLV | 65.1 (6.3) | 64.9 (6.1) | 0.80 |
| % of short telomeres | 3.7 (1.6) | 3.6 (1.6) | 0.67 |
| AG_ratio | 1.3 (0.1) | 1.3 (0.1) | 0.40 |
| Never smokers, N | 11 | 78 | |
| TL | 2614 (487) | 2472 (670) | 0.50 |
| TLV | 62.1 (7.0) | 65.1 (5.9) | 0.12 |
| % of short telomeres | 5.9 (1.6) | 3.8 (1.8) | 1.76 |
| AG_ratio | 1.2 (0.2) | 1.3 (0.1) | 0.37 |
| Former smokers, N | 75 | 95 | |
| TL | 2453 (741) | 2326 (693) | 0.25 |
| TLV | 64.8 (5.3) | 65.8 (6.3) | 0.26 |
| % of short telomeres | 3.5 (1.4) | 3.8 (1.7) | 0.17 |
| AG_ratio | 1.3 (0.1) | 1.3 (0.1) | 0.81 |
| Current smokers, N | 103 | 33 | |
| TL | 2370 (690) | 2502 (746) | 0.35 |
| TLV | 65.4 (6.6) | 62.7 (6.3) | 0.04 |
| % of short telomeres | 3.8 (1.8) | 3.2 (1.7) | 0.14 |
| AG_ratio | 1.3 (0.2) | 1.3 (0.1) | 0.55 |

TL = telomere length expressed as fluorescent intensity units
TLV = telomere length variation, is the co-efficient of variation (CV) of measured telomere lengths
% of short telomeres is the percentage of telomeres that were shorter than 10% of the average telomere length
AG_ratio is the ratio of average TL of A group chromosomes divided by average TL of G group chromosomes.

Table 13 shows data analysis of the association of telomere length to lung cancer risk. The data show significant correlations of telomere length variation and percent of short telomeres to the risk of lung cancer in subject 60 years of age and younger. As before, short telomeres were defined as telomeres that were shorter than 10% of the average telomere length. The data show that, for subjects 60 years old and younger, a telomere length variation over the median telomere length variation (TLV of 65.0) indicates a risk of lung cancer (odds ratio of 6.65 and p=0.0031). The data also show that, for subjects 60 years old and younger, a telomere length variation in the highest quartile (TLV of 69.2-78.7; TLV over 68.7) indicates a risk of lung cancer (odds ratio of 16.06 and p=0.0039). The data also show that, for subjects 60 years old and younger, a percent of short telomeres over the median percent of short telomeres (3.44% of short telomeres; 0.0344 frequency of extremely short telomeres) indicates a risk of lung cancer (odds ratio of 5.11 and p=0.0044). The data also show that, for subjects 60 years old and younger, a percent of short telomeres in the highest quartile (4.89-7.64% of short telomeres; 0.0489-0.0764 frequency of extremely short telomeres) indicates a risk of lung cancer (odds ratio of 25.17 and p=0.0051).

TABLE 13
Risk estimation of association between average TL and lung cancer risk

| | N | | Average TL | | | | N | | TLV | | | | N | | % of short Telomere | | |
|---|---|---|---|---|---|---|---|---|---|---|---|---|---|---|---|---|---|
| TL | cases | controls | OR | 95% CI | p | TLV | cases | controls | OR | 95% CI | p | % short telomere | cases | controls | OR | 95% CI | p |
| All subject | 189 | 206 | | | | All subject | n = 189 | n = 206 | | | | All subject | n = 189 | n = 206 | | | |

by median

| | | | | | | | | | | | | | | | | | |
|---|---|---|---|---|---|---|---|---|---|---|---|---|---|---|---|---|---|
| 1666-4717 | 91 | 106 | ref | | | 48.8-65.2 | 95 | 102 | ref | | | 0.58-3.44 | 95 | 103 | ref | | |
| 4724-9891 | 98 | 100 | 1.51 | 0.94-2.43 | 0.0894 | 65.3-81.5 | 94 | 104 | 0.79 | 0.49-1.28 | 0.3376 | 3.44-9.06 | 94 | 103 | 0.89 | 0.55-1.43 | 0.6298 |

by quartile

| | | | | | | | | | | | | | | | | | |
|---|---|---|---|---|---|---|---|---|---|---|---|---|---|---|---|---|---|
| 1666-3846 | 44 | 54 | ref | | | 48.8-60.7 | 46 | 52 | ref | | | 0.58-2.46 | 49 | 50 | ref | | |
| 3854-4717 | 47 | 52 | 1.45 | 0.73-2.87 | | 60.7-65.2 | 49 | 50 | 1.11 | 0.57-2.17 | | 2.46-3.44 | 46 | 53 | 0.73 | 0.37-1.43 | |
| 4724-5693 | 51 | 48 | 1.88 | 0.95-3.70 | | 65.3-68.9 | 52 | 47 | 1.02 | 0.52-2.01 | | 3.44-4.69 | 50 | 48 | 0.94 | 0.48-1.85 | |
| 5705-9891 | 47 | 52 | 1.78 | 0.90-3.51 | 0.0722 | 68.9-81.5 | 42 | 57 | 0.66 | 0.33-1.34 | 0.2419 | 4.71-9.06 | 44 | 55 | 0.60 | 0.30-1.20 | 0.2539 |
| Age <=60 | n = 54 | n = 61 | | | | Age <=60 | n = 54 | n = 61 | | | | Age <=60 | n = 54 | n = 61 | | | |

by median

| | | | | | | | | | | | | | | | | | |
|---|---|---|---|---|---|---|---|---|---|---|---|---|---|---|---|---|---|
| 1822-4712 | 27 | 30 | ref | | | 48.8-65.0 | 33 | 54 | ref | | | 0.58-3.44 | 22 | 54 | ref | | |
| 4688-8653 | 27 | 31 | 1.14 | 0.48-2.72 | 0.7671 | 65.3-78.7 | 21 | 7 | 6.65 | 1.98-23.36 | 0.0031 | 3.44-7.64 | 32 | 7 | 5.11 | 1.66-15.71 | 0.0044 |

by quartile

| | | | | | | | | | | | | | | | | | |
|---|---|---|---|---|---|---|---|---|---|---|---|---|---|---|---|---|---|
| 1822-3846 | 14 | 20 | ref | | | 48.8-60.6 | 22 | 33 | ref | | | 0.58-2.46 | 22 | 33 | ref | | |
| 3855-4712 | 13 | 10 | 2.57 | 0.68-9.81 | | 60.7-65.0 | 11 | 21 | 1.04 | 0.35-3.05 | | 2.46-3.44 | 10 | 21 | 0.93 | 0.31-2.73 | |
| 4788-5693 | 18 | 9 | 4.70 | 1.26-17.57 | | 65.3-68.7 | 9 | 5 | 3.67 | 0.79-17.05 | | 3.44-4.64 | 12 | 6 | 2.66 | 0.71-9.90 | |
| 5707-8653 | 9 | 22 | 0.67 | 0.18-2.44 | 0.8994 | 69.2-78.7 | 12 | 2 | 16.06 | 2.14-120.4 | 0.0039 | 4.89-7.64 | 10 | 1 | 25.17 | 2.16-292.9 | 0.0051 |
| Age 61-74 | n = 67 | n = 73 | | | | Age 61-74 | n = 67 | n = 73 | | | | Age 61-74 | n = 67 | n = 73 | | | |

by median

| | | | | | | | | | | | | | | | | | |
|---|---|---|---|---|---|---|---|---|---|---|---|---|---|---|---|---|---|
| 2741-4717 | 24 | 28 | ref | | | 54.8-65.2 | 26 | 17 | ref | | | 0.91-3.44 | 29 | 17 | ref | | |
| 4725-8678 | 43 | 45 | 1.24 | 0.45-3.40 | 0.6812 | 65.3-81.5 | 41 | 56 | 0.35 | 0.12-1.01 | 0.0516 | 3.48-9.06 | 38 | 56 | 0.27 | 0.09-0.77 | 0.014 |

by quartile

| | | | | | | | | | | | | | | | | | |
|---|---|---|---|---|---|---|---|---|---|---|---|---|---|---|---|---|---|
| 2741-3842 | 5 | 3 | ref | | | 43.8-60.3 | 11 | 6 | ref | | | 0.91-2.32 | 13 | 4 | ref | | |
| 3854-4717 | 19 | 25 | 0.37 | 0.03-5.37 | | 60.8-65.2 | 15 | 11 | 0.35 | 0.05-2.23 | | 2.46-3.44 | 16 | 13 | 0.23 | 0.03-1.50 | |
| 4725-5657 | 17 | 24 | 0.34 | 0.02-5.03 | | 65.3-68.9 | 21 | 23 | 0.21 | 0.04-1.19 | | 3.48-4.69 | 14 | 26 | 0.07 | 0.01-0.49 | |
| 5705-8678 | 26 | 21 | 0.71 | 0.05-9.80 | 0.4589 | 69.0-81.5 | 20 | 33 | 0.14 | 0.03-0.84 | 0.0272 | 4.75-9.06 | 24 | 30 | 0.12 | 0.02-0.71 | 0.0217 |
| Age >=75 | n = 68 | n = 72 | | | | Age >=75 | n = 68 | n = 72 | | | | Age >=75 | n = 68 | n = 72 | | | |

by median

| | | | | | | | | | | | | | | | | | |
|---|---|---|---|---|---|---|---|---|---|---|---|---|---|---|---|---|---|
| 1666-4680 | 40 | 48 | ref | | | 50.4-65.2 | 36 | 31 | ref | | | 0.72-3.44 | 34 | 32 | ref | | |
| 4724-9891 | 28 | 24 | 2.06 | 0.92-4.64 | 0.0808 | 65.3-80.4 | 32 | 41 | 0.42 | 0.19-0.93 | 0.0325 | 3.48-8.41 | 34 | 40 | 0.67 | 0.32-1.43 | 0.3046 |

by quartile

| | | | | | | | | | | | | | | | | | |
|---|---|---|---|---|---|---|---|---|---|---|---|---|---|---|---|---|---|
| 1666-3784 | 25 | 31 | ref | | | 50.4-60.7 | 13 | 13 | ref | | | 0.72-2.46 | 14 | 13 | ref | | |
| 3897-4680 | 15 | 17 | 1.30 | 0.47-3.61 | | 60.8-65.2 | 23 | 18 | 1.38 | 0.42-4.50 | | 2.53-3.44 | 20 | 19 | 0.49 | 0.15-1.58 | |
| 4724-5667 | 16 | 15 | 2.05 | 0.71-5.96 | | 65.3-68.8 | 22 | 19 | 0.82 | 0.27-2.52 | | 3.48-4.67 | 24 | 16 | 1.04 | 0.32-3.38 | |
| 5716-9891 | 12 | 9 | 2.61 | 0.82-8.30 | 0.0659 | 68.9-80.4 | 10 | 22 | 0.26 | 0.08-0.90 | 0.0161 | 4.71-8.41 | 10 | 24 | 0.17 | 0.05-0.60 | 0.0224 |

odd ratios were adjusted for age, gender, race, smoking status and pack-years

TABLE 14
Association of telomere parameters and host factors, control subjects only

| | | TL* | | TLV | | % short telomeres | | AG_ratio | |
|---|---|---|---|---|---|---|---|---|---|
| Host factors | N | Mean (SD) | p-value | Mean (SD) | p-value | Mean (SD) | p-value | Mean (SD) | p-value |
| Age | | | | | | | | | |
| 40-50 | 22 | 2586 (907) | | 58.7 (3.4) | | 2.0 (0.6) | | 1.26 (0.13) | |
| 51-60 | 38 | 2495 (709) | | 61.3 (4.6) | | 2.6 (1.1) | | 1.30 (0.12) | |
| 61-70 | 58 | 2620 (576) | | 68.3 (5.2) | | 4.5 (1.6) | | 1.31 (0.16) | |
| 71-80 | 65 | 2135 (590) | | 67.0 (5.6) | | 4.4 (1.7) | | 1.28 (0.13) | |
| >=81 | 22 | 2333 (794) | 0.003 | 64.1 (6.1) | <0.001 | 3.5 (1.6) | <0.001 | 1.23 (0.13) | 0.20 |
| Race | | | | | | | | | |
| African American | 78 | 2504 (647) | | 65.3 (6.5) | | 3.9 (1.9) | | 1.31 (0.14) | |
| Caucasian | 128 | 2352 (718) | 0.19 | 64.9 (6.1) | 0.61 | 3.6 (1.6) | 0.25 | 1.27 (0.13) | 0.015 |
| Gender | | | | | | | | | |
| Female | 111 | 2440 (702) | | 65.3 (6.1) | | 3.8 (1.7) | | 1.28 (0.13) | |
| Male | 95 | 2383 (689) | 0.46 | 64.8 (6.3) | 0.61 | 3.7 (1.8) | 0.9 | 1.28 (0.15) | 0.7 |
| Smoking status | | | | | | | | | |
| Never | 78 | 2472 (671) | | 65.1 (5.9) | | 3.8 (1.8) | | 1.28 (0.14) | |
| Former | 95 | 2326 (693) | | 65.8 (6.3) | | 3.8 (1.7) | | 1.29 (0.14) | |
| Current | 33 | 2503 (747) | 0.39 | 62.7 (6.3) | 0.045 | 3.2 (1.7) | 0.12 | 1.28 (0.14) | 0.96 |
| Pack-years | | | | | | | | | |
| <=20 | 61 | 2466 (724) | | 64.7 (6.7) | | 3.5 (1.8) | | 1.28 (0.13) | |
| 21-40 | 34 | 2354 (759) | | 63.7 (6.5) | | 3.5 (1.8) | | 1.27 (0.13) | |
| 41-60 | 22 | 2353 (596) | | 66.9 (4.5) | | 4.2 (1.3) | | 1.31 (0.15) | |
| >=60 | 10 | 1965 (586) | 0.16 | 65.6 (6.4) | 0.29 | 3.8 (1.5) | 0.19 | 1.30 (0.22) | 0.88 |
| Family History of Cancer | | | | | | | | | |
| No | 47 | 2182 (662) | | 64.2 (6.4) | | 3.5 (1.6) | | 1.27 (0.14) | |
| Yes | 159 | 2477 (692) | 0.02 | 65.3 (6.1) | 0.30 | 3.8 (1.8) | 0.25 | 1.29 (0.14) | 0.31 |
| BMI | | | | | | | | | |
| <20 | 14 | 2165 (697) | | 63.6 (4.5) | | 3.2 (1.0) | | 1.24 (0.10) | |
| 20-25 | 67 | 2499 (790) | | 64.3 (6.8) | | 3.5 (1.8) | | 1.31 (0.15) | |
| 25-29 | 88 | 2482 (654) | | 65.4 (5.9) | | 3.9 (1.7) | | 1.26 (0.14) | |
| ≧30 | 44 | 2218 (605) | 0.062 | 65.9 (6.4) | 0.47 | 4.1 (1.9) | 0.16 | 1.29 (0.13) | 0.18 |
| Physical activity, | | | | | | | | | |
| hours/week | | | | | | | | | |
| <=7 | 55 | 2450 (707) | | 63.1 (6.0) | | 3.3 (1.6) | | 1.28 (0.12) | |
| 8-11 | 43 | 2528 (752) | | 65.6 (6.4) | | 3.9 (1.8) | | 1.30 (0.14) | |
| 13-21 | 57 | 2277 (639) | | 66.4 (5.4) | | 4.0 (1.7) | | 1.30 (0.15) | |
| >=22 | 51 | 2414 (688) | 0.49 | 65.1 (6.7) | 0.024 | 3.8 (1.8) | 0.16 | 1.26 (0.16) | 0.16 |

*telomere length in fluorescent intensity units
Means were not adjusted by age

We claim:

1. A method of assaying a subject comprising:
measuring the length of at least one chromosome telomere of a chromosome in at least one cell of a sample from a subject, thereby producing a chromosome telomere length for at least one of the chromosome telomeres,
comparing the chromosome telomere length with a reference chromosome telomere length.

2. The method of claim 1, wherein the chromosome telomere is 1p-S, Xp-S, 9p-S, or 15p-S, wherein the subject is a pre-menopausal female, wherein the subject has an increased risk of breast cancer if the chromosome telomere length is shorter than the reference chromosome telomere length.

3. The method of claim 1, wherein the chromosome telomere is Xp-S or 15p-S, wherein the subject is a pre-menopausal female, wherein the shorter the chromosome telomere length, the greater the risk of breast cancer for the subject.

4. The method of claim 1, wherein the chromosome telomere is 15p-S, wherein the subject is a post-menopausal female, wherein the subject has an increased risk of breast cancer if the chromosome telomere length is shorter than the reference chromosome telomere length.

5. The method of claim 1, wherein the chromosome telomere length is a relative telomere length.

6. The method of claim 1, wherein the chromosome telomere is the shorter telomere of a homologous pair of telomeres, wherein the reference chromosome telomere length is the length of the longer telomere of the homologous pair of telomeres, wherein the subject is a pre-menopausal female, wherein the subject has an increased risk of breast cancer if the homologous telomere length difference (HTLD) is greater in chromosome arm 5q, Xp, 8q, 9p, 12p, 15p, or 15q than a reference homologous telomere length difference (HTLD).

7. The method of claim 6, wherein the reference HTLD is the average, median, or quartile value of the HTLDs in cells from normal subjects of similar type to the cell.

8. The method of claim 1, wherein the chromosome telomere is the shorter telomere of a homologous pair of telomeres, wherein the reference chromosome telomere length is the length of the longer telomere of the homologous pair of telomeres, wherein the subject is a pre-menopausal female, wherein the subject has an increased risk of breast cancer if the homologous telomere length difference (HTLD) is greater in chromosome arm 9p, 15p, or 15q than a reference HTLD.

9. The method of claim 1, wherein the chromosome telomere is the shorter telomere of a homologous pair of telomeres, wherein the reference chromosome telomere length is the length of the longer telomere of the homologous pair of telomeres, wherein the greater the homologous telomere length difference (HTLD) in chromosome arm Xp, 9p, 15p, or 15q, the greater the risk of breast cancer for the subject.

10. The method of claim 1, wherein the length of all of the chromosome telomeres in the cell are measured, wherein the subject is a pre-menopausal female, wherein the subject has an increased risk of breast cancer if the within-cell telomere length variation (WCTLV) is greater than a reference within-cell telomere length variation (WCTLV).

11. The method of claim 10, wherein the reference WCTLV is the average, median, or quartile value of the WCTLV in cells from normal subjects of similar type to the cell.

12. The method of claim 1, wherein the length of all of the chromosome telomeres in the cell are measured, wherein the subject is a pre-menopausal female, wherein the greater the within-cell telomere length variation (WCTLV), the greater the risk of breast cancer for the subject.

13. The method of claim 1, wherein the reference chromosome telomere length is the average, median, or quartile value of the chromosome telomere lengths in cells from normal subjects of similar type to the cell.

14. The method of claim 13, wherein the reference chromosome telomere length is the average, median, or quartile value of the chromosome telomere lengths of the chromosome in cells from normal subjects of similar type to the cell.

15. The method of claim 1, wherein the reference chromosome telomere length is the average, median or quartile value of the arm-specific telomere lengths in cells from normal subjects of similar type to the cell.

16. The method of claim 1, wherein the chromosome telomere length is less than or equal to 0.5 of the reference chromosome telomere lengths.

17. The method of claim 1, wherein measuring the length of at least one chromosome telomere is accomplished by obtaining the sample from the subject, wherein the sample is a blood sample; harvesting the chromosome from at least one cell in the blood sample; performing telomere analysis; and quantitating telomere length.

18. The method of claim 17, wherein performing telomere analysis and quantitating telomere length are accomplished by telomere quantitative fluorescent in situ hybridization (QT-FISH).

19. The method of claim 18, wherein quantitating telomere length is accomplished by totaling the fluorescent signal from telomere probes from the chromosome telomere.

20. The method of claim 1, wherein the length of all of the chromosome telomeres in the cell are measured, wherein the subject is 60 years old or younger, wherein the subject has an increased risk of lung cancer if the within-cell telomere length variation (WCTLV) is greater than a reference within-cell telomere length variation (WCTLV).

21. The method of claim 20, wherein the reference WCTLV is the average, median, or quartile value of the WCTLV in cells from normal subjects of similar type to the cell.

22. The method of claim 21, wherein the reference WCTLV is the highest quartile value of the WCTLV in cells from normal subjects of similar type to the cell.

23. The method of claim 1, wherein the length of all of the chromosome telomeres in the cell are measured, wherein the subject is 60 years old or younger, wherein the greater the within-cell telomere length variation (WCTLV), the greater the risk of lung cancer for the subject.

24. The method of claim 1, wherein the length of all of the chromosome telomeres in the cell are measured, wherein the subject is 60 years old or younger, wherein the subject has an increased risk of lung cancer if the frequency of extremely short telomeres is greater than a reference frequency of extremely short telomeres.

25. The method of claim 24, wherein the reference frequency of extremely short telomeres is the average, median, or quartile value of the frequency of extremely short telomeres in cells from normal subjects of similar type to the cell.

26. The method of claim 24, wherein the reference frequency of extremely short telomeres is the highest quartile value of the frequency of extremely short telomeres in cells from normal subjects of similar type to the cell.

27. The method of claim 1, wherein the length of all of the chromosome telomeres in the cell are measured, wherein the subject is 60 years old or younger, wherein the greater the frequency of extremely short telomeres, the greater the risk of lung cancer for the subject.
